(19) United States
(12) Patent Application Publication
(10) Pub. No.: US 2010/0129430 A1
(43) Pub. Date: May 27, 2010

(54) LIPOSOME DRUG CARRIERS WITH PH-SENSITIVITY

(76) Inventor: Stavroula Sofou, Brooklyn, NY (US)

Correspondence Address: SMITH, GAMBRELL & RUSSELL, SUITE 3100, PROMENADE II, 1230 PEACHTREE STREET, N.E., ATLANTA, GA, 30309-3592, US

(21) Appl. No.: 12/276,696

(22) Filed: Nov. 24, 2008

(51) Int. Cl. A61K 9/127

(52) U.S. Cl. 424/450

(57) ABSTRACT

Using liposomes to deliver bioactive agents to cancer or tumor cells and compositions of specific lipids that form liposomes to deliver a biologically active agent. A liposome composition for delivering of a biologically active agent, comprising liposomes comprising at a least first rigid lipid and a second rigid lipid each having a head group and a hydrophobic tail; a polyethyleneglycol-linked lipid having a side chain matching at least a portion of the first or the second lipid; and an entrapped biologically active agent. The first lipid can be a zwitterionic lipid and the second lipid can have a titratable head group. The composition can be adapted to release the entrapped at a certain pH.

### STATEMENT OF RELATED APPLICATIONS

This application is based on and claims priority on U.S. patent application Ser. No. 11/762,635 having a filing date of 13 Jun. 2007, which is based on and claims priority on U.S. Provisional Patent Application No. 60/804,630 having a filing date of 13 Jun. 2006.

### BACKGROUND OF THE INVENTION

1. Technical Field

This invention generally relates to the field of drug carriers and more particularly relates to the field of using liposomes to deliver biologically active agents to cells, including cancer or tumor cells.

2. Prior Art

Liposomes, which are spherical, self-enclosed vesicles composed of amphipathic lipids, have been widely studied and are employed as vehicles for in vivo administration of therapeutic agents. Liposomes are structures defined by a phospholipid bilayer membrane that encloses an aqueous compartment. The membrane acts as a barrier that inhibits free molecular diffusion across the bilayer. The physicochemical characteristics of the liposome, can be helpful in achieving certain effects from the liposome. Manipulation of these characteristics can have marked effects on the in vivo behavior of liposomes and can have a major impact on therapeutic success.

Liposomes and their potential as drug-delivery vehicles have been investigated for many years. Hydrophilic drugs, such as aminoglycosides, can be encapsulated in the internal aqueous compartments, whereas hydrophobic drugs may bind to or are incorporated in the lipid bilayer. The bilayers are usually composed of natural or synthetic phospholipids and cholesterol, but the incorporation of other lipids or their derivatives, as well as proteins, is also possible.

Accordingly, there is always a need for an improved drug carrier and improved liposome compositions. It is to these needs, among others, that this invention is directed.

### BRIEF SUMMARY OF THE INVENTION

Briefly, this invention includes a liposome composition for delivering a bioactive agent, comprising at a least a first lipid and a second lipid each having a head group; a polyethyleneglycol-linked lipid having a side acyl-chain matching at least a portion of the first lipid or the second lipid; and an entrapped biologically active agent. The composition is adapted to release the entrapped biologically active agent at a certain pH and more specifically to the release of encapsulated contents from liposomes containing lipids with ‘non-matching’ and ‘matching’ hydrocarbon tails.

These features, and other features and advantages of the present invention, will become more apparent to those of ordinary skill in the relevant art when the following detailed description of the preferred embodiments is read in conjunction with the appended drawings in which like reference numerals represent like components throughout the several views.

### BRIEF DESCRIPTION OF THE DRAWINGS

FIG. 1A shows that the liposomes composed of equimolar DPPC and DSPA lipids exhibited a pH-dependent content.

FIG. 1B shows that a decrease in the fraction of the titratable domain-forming lipid DSPA from 50 mol % to 25 mol % resulted in higher content retention at every pH value and every time point studied.

FIG. 1C shows that a decrease in the fraction of the titratable domain-forming lipid DSPA to 10 mol % resulted in higher content retention at every pH value and every time point studied.

FIG. 2A shows the pH-dependent release of fluorescent contents (calcein) from liposomes composed of equimolar DPPC and DSPA lipids in 10% serum supplemented media.

FIG. 2B shows the pH-dependent release of fluorescent contents (calcein) from liposomes composed of 75 mol % DPPC and 25 mol % DSPA lipids in 10% serum supplemented media.

FIG. 2C shows the pH-dependent release of fluorescent contents (calcein) from liposomes composed of 90 mol % DPPC and 10 mol % DSPA lipids in 10% serum supplemented media.

FIG. 2D shows that release of fluorescent contents (calcein) from liposomes composed of lipids with matching acyl-tails (25% DSPA lipid and 75% DSPC lipid) in the presence of 10% serum proteins in media exhibit pH sensitive release of contents.

FIG. 3A shows the content release over 5 days by liposomes composed of 75% mole DPPC and 25% mole DSPA (with 5% mole cholesterol and 2% mole PEGylated lipids), incubated in 60% serum supplemented media at 37° C.

FIG. 3B shows content release upon pH decrease after a 60 minute preincubation period at pH 7.4 by liposomes composed of 75% mole DPPC and 25% mole DSPA (with 5% mole cholesterol and 2% mole PEGylated lipids), incubated in 60% serum supplemented media at 37° C.

FIG. 4A shows the thermal scans of PEGylated liposomes composed of equimolar DPPC and DSPA lipids (with 5% mole cholesterol) in phosphate buffer after two hours of incubation at 37° C.

FIG. 4B shows that longer incubation (four days) of liposome suspensions at 37° C. resulted in similar shifts toward higher thermal transitions with decreasing pH.

FIG. 5 shows the thermographs of equimolar DPPC- and DSPA-containing liposomes at neutral pH in the presence and absence of lysozyme.

FIG. 6 schematically illustrates the formation of heterogeneous domains with decreasing pH from 7.4 to 4.0.

FIG. 7 shows that by using the simplest approximation of formation of small circular phase separated lipid domains of similar size rich in newly protonated DSPA lipids, the rate of content release from liposomes is linearly proportional to the sum of total leaky perimeter of interfacial boundaries that should be proportional to the square root of the number of protonated DSPA lipids.

FIG. 8A shows the content release profiles of the liposome composition containing 21PC and DSPA at about a 75:25 mole ratio with about 10 mol % cholesterol, and about 15 mol % DSPE-PEG(2000) (21PC/DSPA-based liposome) in about 60% serum supplemented media at 37° C.

FIG. 8B shows the content release profiles of the liposome composition containing 21PC and DSPA at about a 75:25 mole ratio with about 10 mol % cholesterol, and about 15 mol % DSPE-PEG(2000) (21PC/DSPA-based liposome) in about 10% serum supplemented media at 37° C.

FIG. 9A shows that DSPC-based liposomes (90 mol % DSPC, 10 mol % cholesterol, and 5 mol % DSPE-PEG(2000)) that are similar to DOXIL®, (Ortho biotech, Bridgewater, N.J.) composition retain at a pH of about 7.4, 5.5 and 5.0 at least about 70% of contents for at least about 24 hours in about 60% serum supplemented media at 37° C.

FIG. 9B shows that DSPC-based liposomes (90 mol % DSPC, 10 mol % cholesterol, and 5 mol % DSPE-PEG(2000)) composition retain at a pH of about 7.4, 5.5 and 5.0 at least about 70% of contents for at least about 24 hours in about 10% serum supplemented media at 37° C.

FIG. 10A shows the percent internalized (immuno)liposomes versus the incubation time as determined by encapsulated calcein.

FIG. 10B shows the release of encapsulated liposome contents in cancer cells.

FIG. 11A shows the release profiles of encapsulated doxorubicin from the liposome composition containing DSPC and cholesterol at about a 9:1 mole ratio with about 5 mol % DSPE-PEG(2000) in about 60% serum supplemented media at 37° C.

FIG. 11B shows the release profiles of encapsulated doxorubicin from the liposome composition containing HSPC and cholesterol at about a 55:45 mole ratio with about 5 mol % DSPE-PEG(2000) in about 60% serum supplemented media at 37° C.

FIG. 11C shows the release profiles of encapsulated doxorubicin from the liposome composition containing 21 PC and DSPA at about a 9:1 mole ratio with about 5 mol % DSPE-PEG(2000) in about 60% serum supplemented media at 37° C.

FIG. 11D shows the release profiles of encapsulated doxorubicin from the liposome composition containing 21PC and DSPA at about a 3:1 mole ratio with about 10 mol % cholesterol, and about 15 mol % DSPE-PEG(2000) in about 60% serum supplemented media at 37° C.

### DEFINITIONS

Unless specifically indicated otherwise, all technical and scientific terms used herein have the same meaning as commonly understood by those of ordinary skill in the art to which this invention belongs. For purposes of the present invention, the following terms are defined.

The term “cancer” as used herein refers to a disease of inappropriate cell proliferation and is more evident when tumor tissue bulk compromises the function of vital organs. Concepts describing normal tissue growth are applicable to malignant tissue because normal and malignant tissues can share similar growth characteristics, both at the level of the single cell and at the level of the tissue.

The term “anionic liposome” as used herein is intended to encompass any liposome as defined below that is anionic. The liposome is determined as being anionic when present in physiological pH. It should be noted that the liposome itself is the entity that is being determined as anionic. The charge and/or structure of a liposome of the invention present within an in vivo environment has not been precisely determined. However, in accordance with the invention an anionic liposome of the invention will be produced using at least some lipids that are themselves anionic. The liposome need not be comprised completely of anionic lipids but must be comprised of a sufficient amount of anionic lipid such that when the liposome is formed and placed within an in vivo environment at physiological pH the liposome initially has a negative charge.

A “pH-sensitive” lipid as used herein refers to a lipid whose ability to change the net charge on its head group depends at least in part on the pH of the surrounding environment.

“Biologically active agents” as used herein refers to molecules which affect a biological system. These include molecules such as proteins, nucleic acids, therapeutic agents, vitamins and their derivatives, viral fractions, lipopolysaccharides, bacterial fractions, and hormones. Other agents of particular interest are chemotherapeutic agents, which are used in the treatment and management of cancer patients. Such molecules are generally characterized as antiproliferative agents, cytotoxic agents, and immunosuppressive agents and include molecules such as taxol, doxorubicin, daunorubicin, vinca-alkaloids, actinomycin, and etoposide.

“Effective amount” as used herein refers to an amount necessary or sufficient to inhibit undesirable cell growth, e.g., prevent undesirable cell growth or reduce existing cell growth, such as tumor cell growth. An effective amount can vary depending on factors known to those of ordinary skill in the art, which include the type of cell growth, the mode and regimen of administration, the size of the subject, the severity of the cell growth. One of ordinary skill in the art would be able to consider such factors and make the determination regarding the effective amount.

“Liposome” as used herein refers to a closed structure comprising an outer lipid bi- or multi-layer membrane surrounding an internal aqueous space. In particular, the liposomes of the present invention form spherical-like structures which invaginate their contents between lipid bilayers. Liposomes can be used to package any biologically active agent for delivery to cells.

The following abbreviations are used herein: PEG: polyethylene glycol; mPEG: methoxy-terminated polyethylene glycol; Chol: cholesterol; DTPA: diethylenetetramine pentaacetic acid; DPPC: dipalmitoylphosphatidylcholine; DSPA: distearoylphosphatidic acid; DSPS: distearoylphosphatidylserine; and 21PC: diheneicosanoyl-phosphocholine.

### DETAILED DESCRIPTION OF PREFERRED EMBODIMENTS

Embodiments of this invention include pH-sensitive liposomes of a specific composition forming a stable structure that can efficiently carry biologically active agents. More particularly, the liposome can contain one or more biologically active agents, which can be administered into a mammalian host to effectively deliver its contents to a target cell or tumor cell. The liposomes are capable of carrying biologically active agents, such that the agents are sequestered in one environment and can be selectively exposed in another.

Liposome Composition

One embodiment of this invention is a pH-sensitive liposome composition for delivering a biologically active agent, comprising:
a) at least a first lipid and a second lipid each having a head group and a hydrophobic tail in the gel state at the working temperature;
b) a polyethyleneglycol-linked lipid having a tail matching at least a portion of the first or the second lipid;
c) cholesterol; and
d) an entrapped biologically active agent.

The composition is adapted to release the entrapped biologically active agent at a certain pH. In one example, one of the lipids is a zwitterionic lipid and one of the lipids is a titratable domain forming lipid. It is understood that additional lipids also can be incorporated into the composition.

Liposomes suitable for use in the composition include those composed primarily of vesicle-forming lipids. Such a vesicle-forming lipid is one that (a) can form spontaneously into bilayer vesicles in water, as exemplified by the phospholipids, or (b) is stably incorporated into lipid bilayers, with its hydrophobic moiety in contact with the interior, hydrophobic region of the bilayer membrane, and its head group moiety oriented toward the exterior, polar surface of the membrane. Many lipids suitable with this embodiment are of the type having two hydrocarbon chains, typically acyl chains, and a head group, either polar or nonpolar. There are a variety of synthetic lipids and naturally forming lipids, including the phospholipids, such as DPPC, DSPA, DPPA, DSPC, and 21PC, where the two hydrocarbon chains are typically at least 16 carbon atoms in length. The vesicle-forming lipids of this type are preferably ones having two hydrocarbon chains, typically acyl chains, and a head group, either polar or nonpolar.

The pH-sensitive liposome can be selected to achieve a specified degree of fluidity or rigidity, to control the stability of the liposome in serum, to control the conditions effective for insertion of the targeting conjugate, as will be described, and to control the rate of release of the entrapped biologically agent in the liposome. Liposomes having a more rigid lipid bilayer, or a gel phase bilayer, are achieved by incorporation of a relatively rigid lipid, e.g., a lipid having a relatively high phase transition temperature, e.g., above about 41° C. Rigid, i.e., saturated, lipids contribute to greater membrane rigidity in the lipid bilayer. Other lipid components, such as cholesterol, are also known to contribute to membrane rigidity in lipid bilayer structures. In contrast, lipid fluidity can be achieved by incorporation of a relatively fluid lipid, typically one having a lipid phase with a relatively low liquid to gel crystalline phase transition temperature, e.g., at or below working temperature (e.g. body temperature).

These liposomes can contain titratable domain-forming lipids that phase-separate in the plane of the membrane as a response to decreasing pH values resulting in pH-controlled release of encapsulated contents. In one embodiment, the liposomes are comprised of two lipid types (both T_{g}'s>37° C.): one type is a zwitterionic rigid lipid (e.g. dipalmitoyl phosphatidyl choline, DPPC, T_{g}=41° C.), and the other component is a titratable domain-forming rigid lipid (e.g. distearoyl phosphatidic acid, DSPA, T_{g}=75° C.) that is triggered to phase-separate in the plane of the membrane as a response to decreasing pH values. At physiological pH (7.4) the lipid-headgroups of the domain-forming rigid lipid are charged, electrostatic repulsion should prevail among DSPA lipids, and the liposomal membrane would appear more mixed and homogeneous, resulting in stable retention of encapsulated contents.

The lipid phase-separation can be tuned by introducing a titratable charge on the headgroups of the domain-forming lipids. The extent of ionization on the headgroups of the domain-forming lipids can be controlled by using the pH to adjust the balance between the electrostatic repulsion among the headgroups and the van der Waals attraction among the hydrocarbon chains. The longer-hydrocarbon chain lipids that could phase-separate and form domains can be selected to have titratable acidic moieties on the headgroup (e.g. phosphatidic acid). At neutral pH, the headgroups of these lipids are negatively charged opposing close approximation and formation of domains. As the pH is decreased, gradual headgroup protonation minimizes the electrostatic repulsion and lipid domains are formed.

In one embodiment, one of the lipids of the liposome can have a negatively charged head group. In this embodiment, the liposomes comprise ionizable ‘domain-forming’ (‘raft’-forming) rigid lipids that are triggered to form domains as a response to the endosomal acidic pH. Domain formation (or else lateral lipid-separation) at the endosomal pH can cause the encapsulated contents to be released probably due to imperfections in ‘lipid packing’ around the domain ‘rim’. At physiological pH (during circulation) the lipids are charged, the liposome membrane may be ‘mixed’ so that the contents cannot leak. At the acidic endosomal pH (5.5-5.0), domain-forming lipids become increasingly protonated (non-ionized) and lipid domains of clustered protonated lipids can form resulting in release of encapsulated contents. In one embodiment, the lipids can have a pK value between about 3 and about 7.0. In another embodiment, the lipids can have a pK value between about 5 and about 5.5.

In another embodiment, the liposomes disclosed herein may further comprise stabilizing agents or have an aqueous phase with a high pH. Examples of stabilizing agents are a phosphate buffer, an insoluble metal binding polymer, resin beads, metal-binding molecules or halogen binding molecules incorporated into the aqueous phase to further facilitate retention of hydrophilic therapeutic modalities. Additionally, liposomes may comprise molecules to facilitate endocytosis by the target cells.

Biologically Active Agents

In one embodiment, the liposomal encapsulation of a biologically active agent enhances the bioavailability of the modalities in cancer cells. In this embodiment, the liposome can be used to encapsulate a biologically active agent (e.g. cancer modalities) and efficiently release the therapeutic modality in cancer cells, thus allowing toxicity to occur in the tumor cells. For example, the use of pH sensitive liposome allows more complete release of the therapeutic modalities upon endocytosis by the cancer cell.

The liposome can have a phospholipid-membrane rigidity to improve the retention of the bioactive agent in the liposome during blood circulation. The additional of PEG also reduces liposome clearance, thus increasing liposome accumulation in tumors. For example, one embodiment includes a pH-sensitive liposome with rigid membranes that combine long circulation times with the release of contents in the endosome. Other types of pH-sensitive liposomes can include charged titratable peptides on the surface that can cause phase separation and domain formation on charged membranes.

Biologically active agents, suitable with such liposomes, include natural and synthetic compounds having the following therapeutic activities: anti-arthritic, anti-arrhythmic, anti-bacterial, anticholinergic, anticoagulant, antidiuretic, antidote, antiepileptic, antifungal, anti-inflammatory, antimetabolic, antimigraine, antineoplastic, antiparasitic, antipyretic, antiseizure, antisera, antispasmodic, analgesic, anesthetic, beta-blocking, biological response modifying, bone metabolism regulating, cardiovascular, diuretic, enzymatic, fertility enhancing, growth-promoting, hemostatic, hormonal, hormonal suppressing, hypercalcemic alleviating, hypocalcemic alleviating, hypoglycemic alleviating, hyperglycemic alleviating, immunosuppressive, immunoenhancing, muscle relaxing, neurotransmitting, parasympathomimetic, sympathominetric plasma extending, plasma expanding, psychotropic, thrombolytic and vasodilating. In one example, the entrapped agent is a cytotoxic drug, that is, a drug having a deleterious or toxic effect on cells.

Administration of Liposome Composition

Another embodiment of this invention includes a method comprising pre-injecting the individual with empty liposomes and saturating the reticuloendothelial organs to reduce non-tumor specific spleen and liver uptake of the liposome-encapsulated therapeutics upon administration thereof.

In use and application, the liposome can be used to preferentially deliver a biologically active agent to a target cell or cancer cell. For example, in drug delivery to metastatic tumors with developed vasculature, the preferential tumor accumulation and retention of liposomes is primarily dependent on their size (EPR effect), and can result in adequate tumor adsorbed doses.

The liposome of the invention may be formulated for parenteral administration by bolus injection or continuous infusion. Formulation for injection may be presented in unit dosage form in ampoules, or in multi-dose containers with an added preservative. The compositions may take such forms as suspensions, solutions or emulsions in oily or aqueous vehicles, and may contain formulatory agents such as suspending, stabilizing, and/or dispersing agents. Alternatively, the active ingredient may be in powder form for reconstitution with a suitable vehicle, e.g. sterile pyrogen-free water, before use.

One embodiment includes a method for administering a biologically active agent comprising selecting a liposome comprising at least a first rigid lipid and a second rigid lipid each having a head group and a hydrophobic tail and a polyethyleneglycol-linked lipid having a side chain matching at least a portion of the first or the second lipid, wherein the first lipid is a zwitterionic lipid and the second lipid has a titratable head group; and the composition is adapted to release the entrapped biologically active agent at a certain pKa; preparing a liposome composition with the at least the first rigid lipid and the second rigid lipid and the polyethyleneglycol-linked lipid; preparing a therapeutic liposome by combining the composition with a biologically active agent so that the biologically active agent is within the liposome composition whereby the therapeutic liposome is adapted to release the entrapped biologically active agent at a certain pKa; and administering the therapeutic liposome to a subject.

The liposomes according to the invention may be formulated for administration in any convenient way. The invention therefore includes within its scope pharmaceutical compositions comprising at least one liposomal compound formulated for use in human or veterinary medicine. Such compositions may be presented for use with physiologically acceptable carriers or excipients, optionally with supplementary medicinal agents. Conventional carriers can also be used with the present invention.

Overcoming Immune Response

To overcome immunogenicity, the liposomes can be modified for use with the specific organism by those with ordinary skill in the art. In another embodiment, a method further comprises coating the outer membrane surfaces of the liposomes with molecules that preferentially associate with a specific target cell. These molecules or targeting agents may be antibodies, peptides, engineered molecules, or fragments thereof.

For example, to achieve tumor targeting of ovarian and breast cancer cells and internalization, liposomes can be coated (immunolabeled) with Herceptin, a commercially available antibody that targets antigens that are overexpressed on the surface of such cancer cells. Herceptin is chosen to demonstrate proof of principle with the anticipation that other antibodies, targeting ovarian, breast, liver, colon, prostate and other carcinoma cells, could also be used. The target cells may be cancer cells or any other undesirable cell. Examples of such cancer cells are those found in ovarian cancer, breast cancer or metastatic cells thereof. The active targeting of liposomes to specific organs or tissues can be achieved by incorporation of lipids with monoclonal antibodies or antibody fragments that are specific for tumor associated antigens, lectins, or peptides attached thereto.

Because the biologically active agent is sequestered in the liposomes, targeted delivery is achieved by the addition of peptides and other ligands without compromising the ability of these liposomes to bind and deliver large amounts of the agent. The ligands are added to the liposomes in a simple and novel method. First, the lipids are mixed with the biologically active agent of interest. Then ligands are added directly to the liposomes to decorate their exterior surface.

Preparing Liposomes

The liposomes may be prepared by a variety of techniques, such as those detailed in Lasic, D. D. Liposomes from Physics to Applications, Elsevier, Amsterdam (1993), and specific examples of liposomes prepared in support of the present invention will be described herein. Typically, the liposomes can be formed by simple lipid-film hydration techniques. In this procedure, a mixture of liposome-forming lipids of the type detailed above dissolved in a suitable organic solvent is evaporated in a vessel to form a thin film, which is then covered by an aqueous medium. The lipid film hydrates have sizes between about 0.1 to 10 microns.

After formation, the liposomes are sized. One more effective sizing method for liposomes involves extruding an aqueous suspension of the liposomes through a series of polycarbonate membranes having a selected uniform pore size in the range of 0.03 to 0.2 micron, typically about 0.05, 0.08, 0.1, or 0.2 microns. The pore size of the membrane corresponds roughly to the largest sizes of liposomes produced by extrusion through that membrane, particularly where the preparation is extruded two or more times through the same membrane. Homogenization methods are also useful for down-sizing liposomes to sizes of 100 nm or less. In one embodiment of the present invention, the liposomes are extruded through a series of polycarbonate filters with pore sizes ranging from 0.2 to 0.08 μm resulting in liposomes having diameters in the approximate range of about 120 nm.

Incorporating Biologically Active Agent into Liposomes

The biologically active agent of choice can be incorporated into liposomes by standard methods, including passive entrapment of a water-soluble compound by hydrating a lipid film with an aqueous solution of the agent, passive entrapment of a lipophilic compound by hydrating a lipid film containing the agent, and loading an ionizable drug against an inside/outside liposome pH gradient. Other methods, such as reverse evaporation phase liposome preparation, are also suitable.

Another embodiment includes a method of formulating a therapeutic liposome composition having sensitivity to a target cell. The method includes selecting a liposome formulation composed of pre-formed liposomes having an entrapped biologically active agent; selecting from a plurality of targeting conjugates a targeting conjugate composed of a lipid having a polar head group and a hydrophobic tail, a hydrophilic polymer having a proximal end and a distal end, and a targeting ligand attached to the distal end of the polymer; and combining the liposome formulation and the selected targeting conjugate to form a therapeutic, target-cell pH sensitive liposome composition.

The following examples serve to illustrate further the present invention.

### EXAMPLES

In the following examples, the constituent lipids were selected to both have long saturated hydrocarbon-chains of different lengths, the shorter being mostly in the gel phase (T_{g}=41° C.) and the longer being in the gel phase (T_{g}=75° C.) at the working temperature (37° C.), so that domain formation would have persistent lipid packing ‘defects’ along the domain/non-domain interface and cause increased membrane permeability and release of encapsulated contents. The choice of lipids having high T_{g} values was thought to be more useful in drug delivery applications.

The lipids for use in these examples include 1,2-Dipalmitoyl-sn-Glycero-3-Phosphocholine (DPPC), 1,2-Dipalmitoyl-sn-Glycero-3-Phosphate (Monosodium Salt) (DPPA), 1,2-Distearoyl-sn-Glycero-3-Phosphocholine (DSPC), 1,2-Distearoyl-sn-Glycero-3-Phosphate (Monosodium Salt) (DSPA), 1,2-Dilauroyl-sn-Glycero-3-Phosphocholine (DLPC), 1,2-Dipalmitoyl-sn-Glycero-3-Phosphoethanolamine-N-[Methoxy(Polyethylene glycol)-2000] (Ammonium Salt) (DPPE-PEG), 1,2-Distearoyl-sn-Glycero-3-Phosphoethanolamine-N-[Methoxy(Polyethylene glycol)-2000] (Ammonium Salt) (DSPE-PEG), 1,2-Dipalmitoyl-sn-Glycero-3-Phosphoethanolamine-N-(Lissamine Rhodamine B Sulfonyl) (Ammonium Salt) (rhodamine-lipid), 1,2-Dioleoyl-sn-Glycero-3-Phosphoethanolamine-N-(7-nitro-2-1,3-benzoxadiazol-4-yl) (Ammonium Salt) (NBD-lipid), and 1,2-Diheneicosanoyl-sn-Glycero-3-Phosphocholine (21 PC). These lipids were purchased from traditional sources.

Using select lipids, the liposomes were prepared by first combining the lipids in chloroform (DPPC and DSPA, or DPPC and DPPA, or DSPC and DSPA, with 5% mole cholesterol, and 2% total PEGylated lipids, DPPE-PEG and DSPE-PEG at 2% mole fractions of the dipalmitoyl- and distearoyl-lipid amounts, respectively) in a 25 ml round bottom flask. The chloroform was evaporated in a rotavapor for 10 minutes at 55° C. and further evaporated under a N₂ stream for 5 minutes. The dried lipid film was then hydrated in 1 ml phosphate buffer (PBS with 1 mM EDTA, pH=7.4) for 2 hours at 50° C. The lipid suspension (10 μmoles total lipid/ml) was then extruded 21 times through two stacked polycarbonate filters of 100 nm pore diameter. Extrusion was carried at 80° C. in a water bath or 5° C. above the highest T_{g} of the constituent lipids.

In order to evaluate the extent of lipid phase-separation at different pH values, differential scanning calorimetry studies were performed using a VP-DSC Instrument (MicroCal, LLC, Northampton, Mass.). DSC scans of liposome suspensions (0.5 ml, 2.5 mM total lipid) with encapsulated phosphate buffer at the same pH as the surrounding phosphate buffer solution (7.4, 5.5, and 4.0) were performed from 10° C. to 85° C. at a scan rate of 60° C./hr. The thermograph of the corresponding buffer was also acquired at identical conditions, and was subtracted from the excess heat capacity curves. The same scanning conditions were used for the studies with externally added lysozyme (at 3.12 mg protein/ml). Liposome suspensions with encapsulated phosphate buffer (pH 7.4) were incubated for two hours at 37° C. in the presence or absence of lysozyme in solutions of different pH values (7.4, 5.5, and 4.0) before acquisition of thermographs.

### Example 1

The release of encapsulated fluorescent contents, specifically in this example calcein, from PEGylated liposomes, composed of different fractions of DPPC and DSPA was investigated by calcein quenching efficiency measurements. The lipid film was hydrated in 1 ml phosphate buffer containing 55 mM calcein (pH 7.4, isomolar to PBS). The unentrapped calcein was removed at room temperature by size exclusion chromatography (SEC) using a Sephadex G-50 column (of 11 cm length) and was eluted with phosphate buffer (1 mM EDTA, pH=7.4). To evaluate the release of calcein from the liposomes, the liposomes containing self-quenching concentrations of calcein (55 mM) were incubated in phosphate buffer or serum supplemented media at different pH values at 37° C. over time. The concentration of lipids for incubation was 0.20 μmoles/ml.

The release of calcein from the liposomes and its dilution in the surrounding solution resulted in an increase in fluorescence due to relief of self-quenching. Calcein release was measured at different time points by adding fixed quantities of liposome suspensions into cuvettes (1 cm path length) containing phosphate buffer (1 mM EDTA, pH 7.4). Calcein fluorescence (ex: 495 nm, em: 515 nm) before and after addition of Triton-X 100, was measured using a Fluoromax-2 spectrofluorometer (Horiba Jobin Yvon, NJ), and was used to calculate the quenching efficiency defined as the ratio of fluorescence intensities after and before addition of Triton-X 100. The percentage of retained contents with time was calculated as follows:

$$\% calcein content retention = ( \frac{{Q}_{t} - {Q}_{min}}{{Q}_{max} - {Q}_{min}} ) \times 100$$

where, Qₜ is calcein quenching efficiency at the corresponding time point t, Qₘₐₓ is the maximum calcein quenching efficiency in phosphate buffer (at pH 7.4) at room temperature immediately after separation of liposomes by SEC, and Qₘᵢₙ is the minimum quenching efficiency equal to unity.

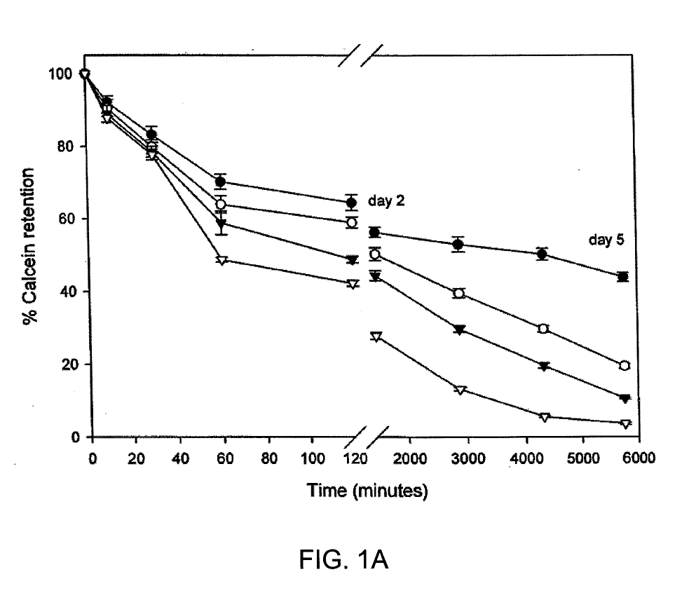

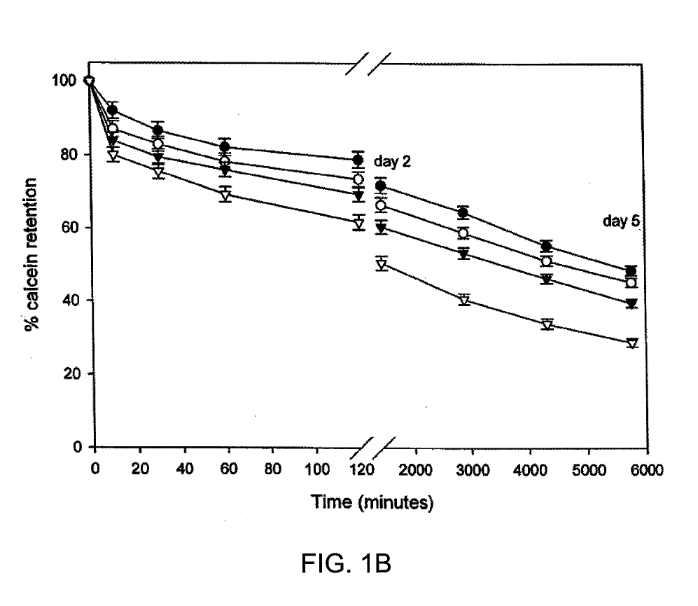

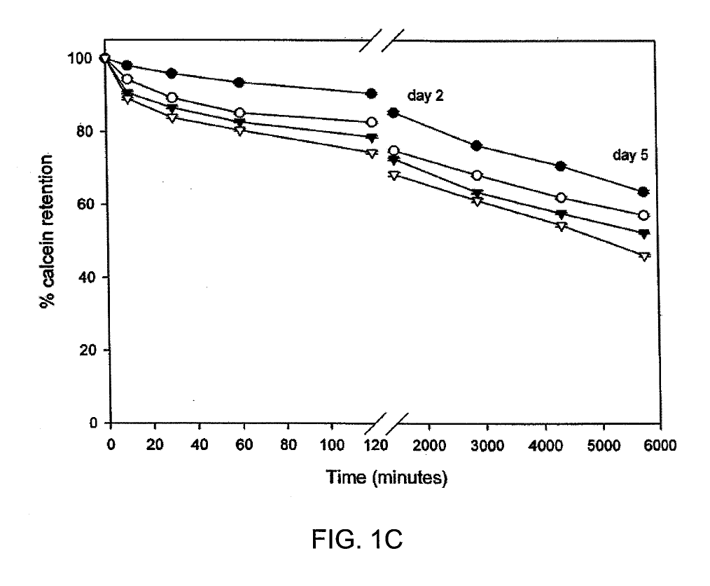

FIGS. 1A, 1B and 1C show that the liposome compositions exhibited pH-dependent behavior as demonstrated by increased content release with decreasing pH in PBS [pH 7.4 (), pH 5.5 (◯), pH 5.0 (▾), pH 4.0 (∇)]. As shown in FIG. 1A, the liposomes composed of equimolar DPPC and DSPA lipids exhibited the faster pH-dependent content release. FIGS. 1B and 1C show that a decrease in the fraction of the titratable domain-forming lipid DSPA from 50 mol % to 25 mol % and 10 mol %, respectively, resulted in higher content retention at every pH value and every time point studied. After 30 days of incubation, the contents were completely released from all liposome compositions. PEGylated liposomes composed only of DPPC lipids or only of DSPA lipids exhibited stable retention of 60% or 80%, respectively, of encapsulated contents in phosphate buffer that was not pH-dependent.

Measurable and similar content release (approximately 5 to 10%) was detected within the first 10 minutes of liposome incubation at 37° C. for all liposome compositions and pH values studied. Release of contents during this interval, in addition to phase separation, could due to differences in osmolarity between the encapsulated solution and the surrounding solvent, or due to the fast heating of liposomes from room temperature to 37° C. that is close to the T_{g} value of the DPPC liposome component (T_{g}=41° C.) resulting in melting of defects in the DPPC-rich phase due to formation of transient boundary regions between liquid and solid domains.

Release of encapsulated contents from liposomes containing ‘matching’ hydrocarbon tails (DPPC and DPPA lipids, and DSPC and DSPA lipids) in PBS is shown on Tables 1A and 1B, respectively.

TABLE 1A

| | 50% mole DPPA | | | | 25% mole DPPA | | | |
|---|---|---|---|---|---|---|---|---|
| Time | pH | | | | pH | | | |
| (min.) | 7.4 | 5.5 | 5.0 | 4.0 | 7.4 | 5.5 | 5.0 | 4.0 |
| 0 | 100 ± 0 | 100 ± 0 | 100 ± 0 | 100 ± 0 | 100 ± 0 | 100 ± 0 | 100 ± 0 | 100 ± 0 |
| 10 | 69 ± 3 | 66 ± 1 | 64 ± 2 | 59 ± 2 | 86 ± 4 | 80 ± 3 | 86 ± 3 | 80 ± 4 |
| 30 | 64 ± 2 | 63 ± 2 | 58 ± 2 | 59 ± 2 | 86 ± 4 | 77 ± 2 | 85 ± 4 | 80 ± 4 |
| 60 | 61 ± 2 | 61 ± 4 | 56 ± 1 | 60 ± 2 | 79 ± 5 | 76 ± 1 | 79 ± 3 | 79 ± 6 |
| 120 | 61 ± 8 | 62 ± 2 | 57 ± 4 | 55 ± 2 | 78 ± 4 | 76 ± 4 | 76 ± 4 | 77 ± 4 |
| 1440 | 47 ± 3 | 41 ± 1 | 41 ± 1 | 35 ± 1 | 64 ± 1 | 59 ± 1 | 61 ± 2 | 58 ± 1 |
| (day 2) | | | | | | | | |
| 5760 | 28 ± 1 | 20 ± 1 | 22 ± 1 | 14 ± 1 | 36 ± 2 | 28 ± 2 | 29 ± 4 | 27 ± 6 |
| (day 5) | | | | | | | | |

TABLE 1B

| | 50% mole DSPA | | | | 25% mole DSPA | | | |
|---|---|---|---|---|---|---|---|---|
| Time | pH | | | | pH | | | |
| (min.) | 7.4 | 5.5 | 5.0 | 4.0 | 7.4 | 5.5 | 5.0 | 4.0 |
| 0 | 100 ± 0 | 100 ± 0 | 100 ± 0 | 100 ± 0 | 100 ± 0 | 100 ± 0 | 100 ± 0 | 100 ± 0 |
| 10 | 69 ± 2 | 63 ± 1 | 64 ± 3 | 56 ± 2 | 89 ± 4 | 87 ± 6 | 86 ± 3 | 76 ± 3 |
| 30 | 62 ± 1 | 64 ± 1 | 57 ± 2 | 49 ± 1 | 83 ± 3 | 83 ± 3 | 78 ± 3 | 74 ± 3 |
| 60 | 62 ± 4 | 58 ± 1 | 61 ± 1 | 51 ± 1 | 75 ± 3 | 68 ± 6 | 66 ± 2 | 71 ± 5 |
| 120 | 63 ± 1 | 58 ± 5 | 49 ± 3 | 50 ± 4 | 72 ± 2 | 63 ± 3 | 69 ± 3 | 66 ± 5 |
| 1440 | 53 ± 1 | 46 ± 4 | 37 ± 0 | 32 ± 1 | 67 ± 3 | 66 ± 2 | 63 ± 2 | 62 ± 2 |
| (day 2) | | | | | | | | |
| 5760 | 39 ± 1 | 17 ± 1 | 13 ± 1 | 13 ± 0 | 60 ± 2 | 59 ± 1 | 58 ± 3 | 52 ± 2 |
| (day 5) | | | | | | | | |

As can be seen, both types of liposomes exhibited an initial drop in content retention within the first 10 minutes of incubation that was dependent on the fraction of ‘titratable lipid’ (DPPA or DSPA, Table 1A and Table 1B, respectively) and not on pH. Decrease of the fraction of DPPA or DSPA lipid resulted in higher overall content retention. For the first 24 hours of incubation, all liposome compositions exhibited content release profiles that were not strongly pH-dependent.

TABLE 2

| | Percentage of calcein retention | | | |
|---|---|---|---|---|
| | pH | | | |
| Time (min.) | 7.4 | 5.5 | 5.0 | 4.0 |
| 0 | 100 ± 0 | 100 ± 0 | 100 ± 0 | 100 ± 0 |
| 10 | 92 ± 2 | 85 ± 3 | 86 ± 2 | 82 ± 2 |
| 30 | 86 ± 3 | 80 ± 3 | 79 ± 4 | 73 ± 2 |
| 60 | 81 ± 2 | 74 ± 2 | 71 ± 4 | 66 ± 2 |
| 120 | 78 ± 3 | 71 ± 2 | 69 ± 3 | 62 ± 3 |
| 1440 (day 2) | 58 ± 2 | 53 ± 3 | 49 ± 3 | 37 ± 2 |
| 5760 (day 5) | 30 ± 4 | 25 ± 3 | 23 ± 6 | 13 ± 9 |

### Example 2

For dextran retention measurements, the dried lipid film was hydrated with 1 ml phosphate buffer containing 3 kDa dextrans (0.5 mg/ml) or 10 kDa dextrans (0.62 mg/ml). To evaluate the release of dextrans from liposomes, liposomes containing fluorescent dextrans of variable molecular weight (3 kDa and 10 kDa) were incubated in phosphate buffer at different pH values at 37° C. over time. The concentration of lipids during incubation was 1.25 μmoles/ml in phosphate buffer in order to increase the concentration of encapsulated dextrans and to improve the efficacy of their detection due to low dextran entrapment efficiency by liposomes. At various time points, liposome fractions were removed from the parent liposome suspension and released dextrans were separated from liposomes by SEC using a Sepharose 4B column (of 11 cm length) eluted with phosphate buffer (1 mM EDTA, pH=7.4) at room temperature. Free dextrans and dextrans encapsulated in liposomes were quantitated by fluorescence spectroscopy (ex: 595 nm, em: 615 nm).

Smaller dextrans (MW=3 KDa, approximately 4.4 nm in diameter) were released in a pH-dependent manner only from liposomes that contained the maximum fraction (50% mole) of DSPA lipid. Large dextran particles (MW=10 KDa, approximately 5.7 nm in diameter) were not released from any composition at all conditions studied indicating an upper cutoff size of the domain/non-domain interface.

To evaluate the extent of liposomal membrane ‘discontinuities’ caused by lipid-phase separation with decreasing pH-values, fluorescently labeled dextrans of molecular sizes larger than calcein, were encapsulated in liposomes and their pH-dependent release from liposomes was measured.

Table 3 shows that 3 kDa dextrans are released from equimolar DPPC and DSPA liposomes in a pH-dependent manner with comparable kinetics to those of calcein. Decreasing the fraction of the titratable domain-forming lipid DSPA (25% and 10% mole DSPA content, Table 3) increased retention of contents and exhibited almost loss of pH-dependent content release. Larger dextrans (10 kDa) were stably retained (>93%) by all liposome compositions for 30 days (data not shown).

TABLE 3

| | 50% mole DSPA | | | | 25% mole DSPA | | | |
|---|---|---|---|---|---|---|---|---|
| Time | pH | | | | pH | | | |
| (min.) | 7.4 | 5.5 | 5.0 | 4.0 | 7.4 | 5.5 | 5.0 | 4.0 |
| 0 | 100 ± 0 | 100 ± 0 | 100 ± 0 | 100 ± 0 | 100 ± 0 | 100 ± 0 | 100 ± 0 | 100 ± 0 |
| 10 | 95 ± 2 | 85 ± 3 | 86 ± 3 | 79 ± 4 | 97 ± 4 | 99 ± 0 | 98 ± 1 | 94 ± 1 |
| 30 | 93 ± 0 | 81 ± 4 | 80 ± 2 | 71 ± 4 | 98 ± 1 | 94 ± 10 | 93 ± 8 | 93 ± 5 |
| 60 | 89 ± 1 | 79 ± 3 | 75 ± 4 | 68 ± 2 | 96 ± 2 | 98 ± 1 | 97 ± 0 | 95 ± 1 |
| 1440 | 88 ± 2 | 68 ± 3 | 66 ± 2 | 61 ± 2 | 97 ± 2 | 94 ± 6 | 95 ± 2 | 93 ± 6 |
| (day 2) | | | | | | | | |
| 5760 | 68 ± 2 | 59 ± 3 | 60 ± 3 | 51 ± 3 | 95 ± 2 | 94 ± 2 | 93 ± 1 | 90 ± 3 |
| (day 5) | | | | | | | | |

| | |
|---|---|
| | 10% mole DSPA |
| Time | pH |

| (min.) | 7.4 | 5.5 | 5.0 | 4.0 |
|---|---|---|---|---|
| 0 | 100 ± 0 | 100 ± 0 | 100 ± 0 | 100 ± 0 |
| 10 | 95 ± 3 | 97 ± 1 | 98 ± 1 | 96 ± 2 |
| 30 | 90 ± 3 | 93 ± 2 | 95 ± 2 | 94 ± 6 |
| 60 | 91 ± 2 | 94 ± 4 | 97 ± 0 | 95 ± 1 |
| 1440 | 93 ± 2 | 95 ± 3 | 96 ± 4 | 96 ± 2 |
| (day 2) | | | | |
| 5760 | 91 ± 4 | 94 ± 4 | 96 ± 1 | 94 ± 0 |
| (day 5) | | | | |

### Example 3

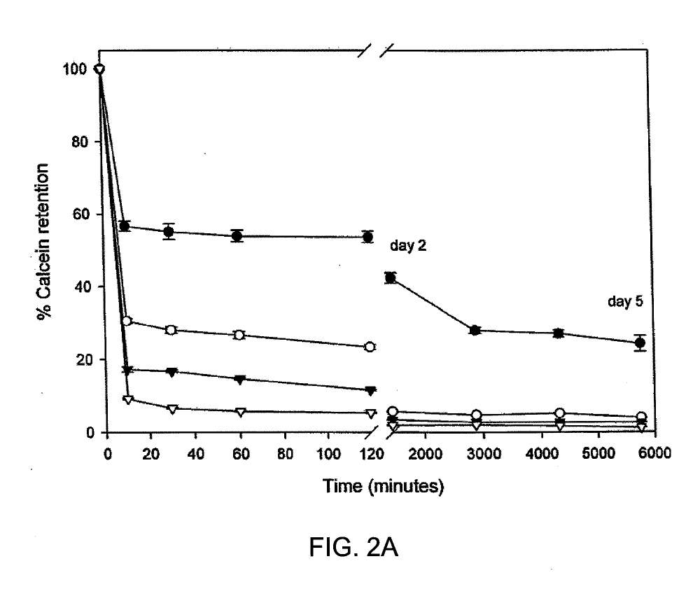

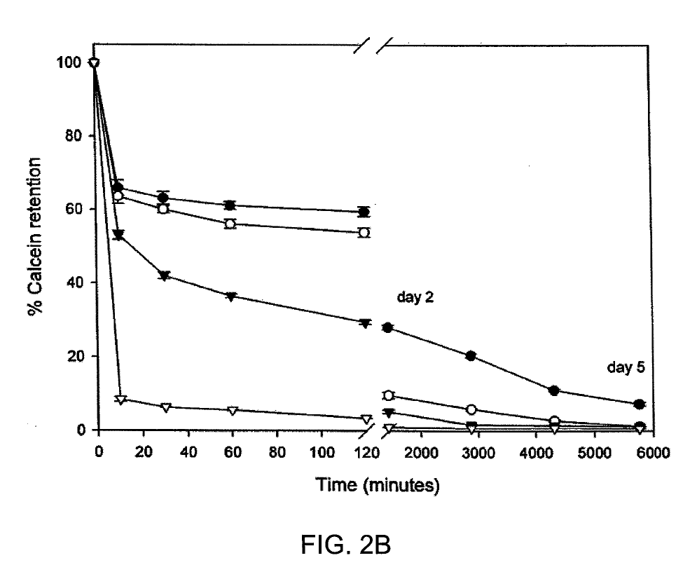

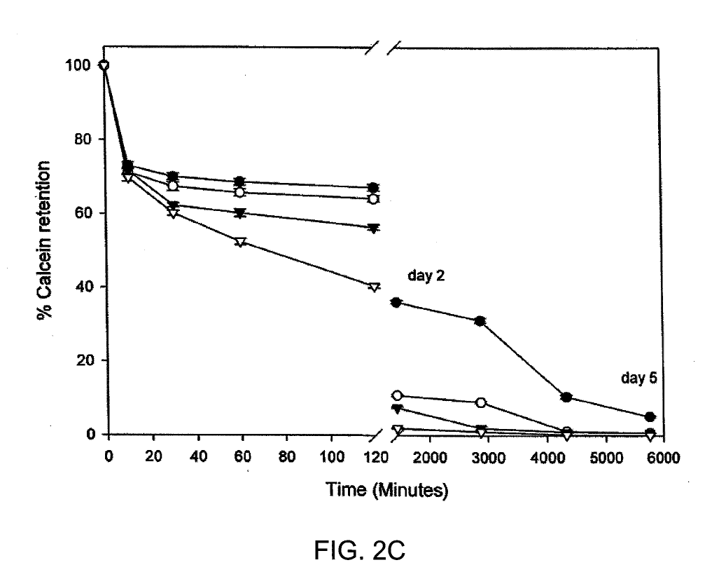

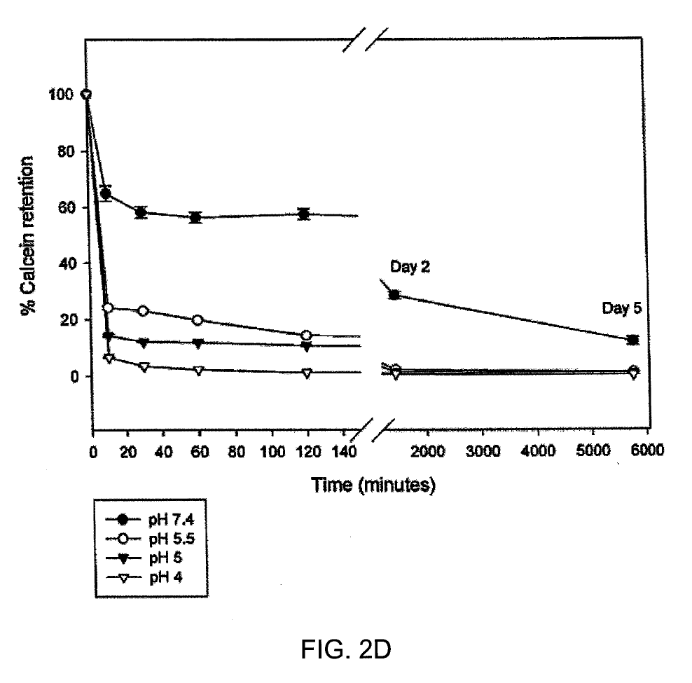

FIGS. 2A, 2B and 2C show the pH-dependent release of fluorescent contents (calcein) from liposomes composed of DPPC and DSPA lipids (1:1, 2:1, 9:1 mole ratios, respectively) in 10% serum supplemented media. Liposomes containing larger fractions of the titratable domain-forming lipid DSPA exhibited greater and faster release of contents, at every pH value studied [pH 7.4 (), pH 5.5 (◯), pH 5.0 (▾), pH 4.0 (∇)]. Release kinetics was significantly faster compared to measurements in phosphate buffer. In particular, liposomes containing equimolar DPPC and DSPA lipids released within 30 minutes of incubation at the ‘endosomally relevant’ pH values of 5.5 and 5.0, 49% and 70% of their contents, respectively, compared to pH 7.4. The observed content release within the first 10 minutes of incubation appears to be pH-dependent in addition to a possible thermal or osmotic effect on membrane destabilization at the onset of incubation as mentioned above.

### Example 4

FIG. 2D shows that release of fluorescent contents (calcein) from liposomes composed of lipids with matching hydrocarbon tails (25% DSPA lipid and 75% DSPC lipid) in the presence of 10% serum proteins in media exhibit pH sensitive release of contents. In less than 20 minutes in acidic pH (5.5 and 5.0, corresponding to early and late endosomal environments), these liposomes release a very significant fraction of the encapsulated contents [pH 7.4 (), pH 5.5 (◯), pH 5.0 (▾), pH 4.0 (∇)]. At the same time, these liposomes stably contain their contents at pH 7.4 that corresponds to the pH during blood circulation where contents are required to be retained by the liposomes.

### Example 5

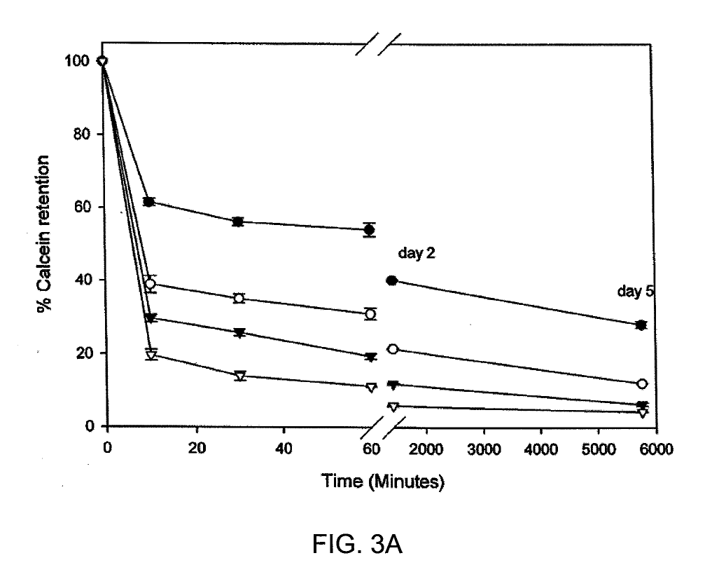

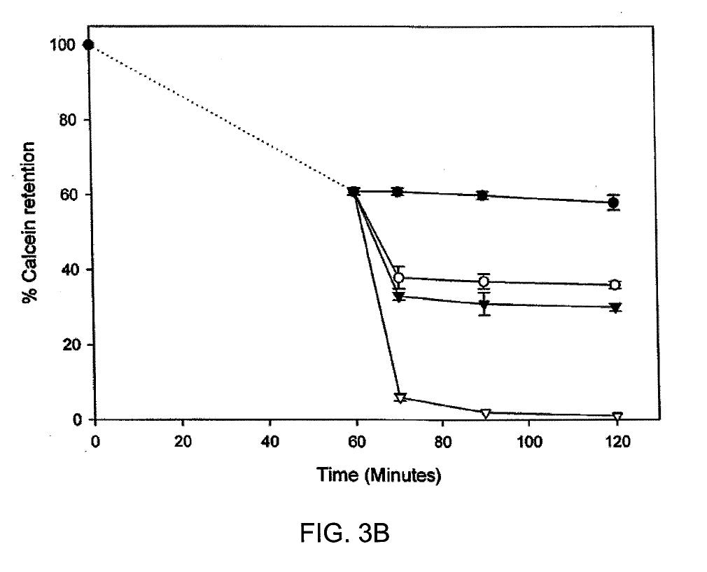

FIGS. 3A and 3B shows the percentage of calcein retention as a function of pH [pH 7.4 (), pH 5.5 (◯), pH 5.0 (▾), pH 4.0 (∇)] by liposomes composed of 75% mole DPPC and 25% mole DSPA (with 5% mole cholesterol and 2% mole PEGylated lipids), incubated in 60% serum supplemented media at 37° C. FIG. 3A shows the content release over 5 days and FIG. 3B shows content release upon pH decrease after a 60 minute preincubation period at pH 7.4. The error bars correspond to standard deviations of repeated measurements 2 liposome preparations, 2 samples per preparation per time point.

FIG. 3A shows that release of fluorescent contents (calcein) from liposomes (25% mole DSPA lipid and 75% DPPC) in the presence of physiologic serum concentrations (60% serum at 37 degrees) exhibit similar dependence on pH as in low serum containing media [pH 7.4 (), pH 5.5 (◯), pH 5.0 (▾), pH 4.0 (∇)]. For short incubation times (less than one hour), liposomes exhibited increased content release at lower pH values. pH-sensitivity was observed in all three lipid ratios studied (data not shown for 50% and 10% mole DSPA). The extent of released contents and the initial release rates in serum supplemented media increase with decreasing pH and with increasing serum concentration.

### Example 6

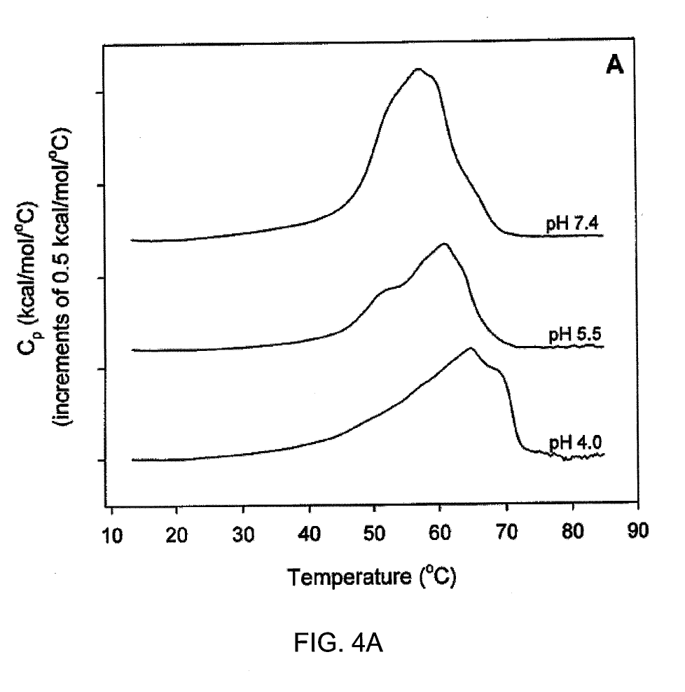

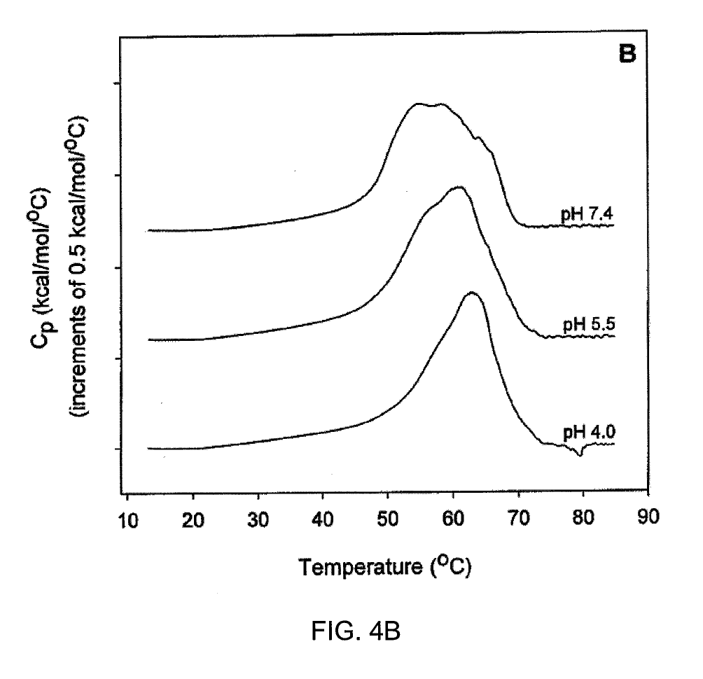

FIG. 4A shows the thermal scans of PEGylated liposomes composed of equimolar DPPC and DSPA lipids (with 5% mole cholesterol) in phosphate buffer after two hours of incubation at 37° C. Enhancement on the contributions from thermal transitions at higher temperatures is observed with decreasing pH values from 7.4 to 4.0. Higher thermal transitions containing multiple peaks at lower pH values suggest increasing formation of lipid phases that should be rich in clustered (newly protonated) DSPA lipids. FIG. 4B shows that longer incubation (four days) of liposome suspensions at 37° C. resulted in similar shifts toward higher thermal transitions with decreasing pH, but with increased calorimetric peaks compared to the earlier time point (FIG. 4A). Decrease of the fraction of the titratable domain-forming lipid DSPA (25% and 10% mole) resulted in similar contributions from thermal transitions but at lower temperatures for all pH values studied (7.4, 5.5, and 4.0).

Lipid phase-separation in the presence of serum should be caused by clustering of charged DSPA lipids that form domains under the electrostatic attraction of charged serum proteins that are adhering onto the liposome membranes. In serum supplemented media, a similar mechanism of domain formation should account for the observed pH-sensitivity of the liposomes studied. Although liposome aggregation was detected with decreasing solution pH and with time, no fusion was detected among the aggregated liposomes. In 60% serum supplemented media, liposomes containing 25% mole DSPA lipids exhibited pH-dependent release of contents and they significantly retained their encapsulated contents at pH 7.4 even after 24 hours of incubation. The pH-sensitive release kinetics is fast and comparable to the kinetics of endocytosis. This is supported by the DSC thermographs using lysozyme as a model protein.

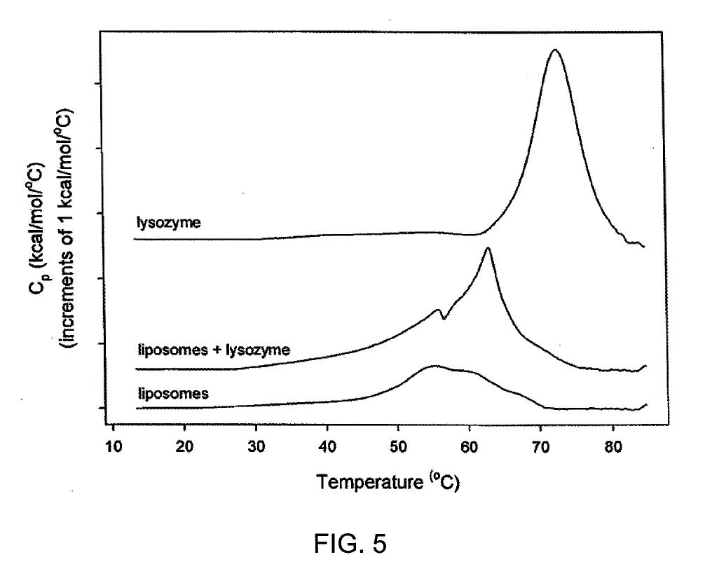

In the presence of serum proteins the pH-dependent content release from liposomes should be due to the phase separation of charged DSPA lipids caused by adsorption of charged proteins onto the liposome surface. To test this hypothesis, lysozyme was added to liposome suspensions and the thermal transitions were evaluated. Lysozyme was used as a model protein because of its high denaturation temperature (72° C.) that should not significantly interfere with the temperature window defined by the thermal transitions of DPPC and DSPA lipids (41° C. and 75° C., respectively). FIG. 5 shows the thermographs of equimolar DPPC- and DSPA-containing liposomes at neutral pH in the presence and absence of lysozyme. The split of the main transition peak of liposomes in the presence of lysozyme probably suggests lateral phase separation caused by the deprotonated (negatively charged) DSPA lipids upon protein adsorption. The shift of the protein denaturation peak to lower temperatures also suggests adsorption of lysozyme onto the lipid membrane. Thermographs at lower pH values (5.5 and 4.0) in the presence of lysozyme resulted in multiple splits on the broad lipid transition peak that could probably be correlated to the more than one transition temperatures that were detected on free lysozyme at the corresponding pH values (data not shown).

### Example 7

The measured average liposome size of equimolar DPPC- and DSPA-containing liposomes was 262±57 nm in diameter at pH 7.4 immediately after liposome extrusion in phosphate buffer. Table 4 shows that incubation of liposomes at 37° C. in phosphate buffer at pH values ranging from 7.4 to 4.0, does not cause changes on the liposome size distributions over time (three days), suggesting that no liposome aggregation occurs. As shown in Table 4, in 10% serum supplemented media, liposome aggregation was observed at the lower pH values and increased with time and with increasing DSPA content.

TABLE 4

| | 50% mole DSPA | | | 25% mole DSPA | | | 10% mole DSPA | | |
|---|---|---|---|---|---|---|---|---|---|
| Time | pH | | | pH | | | pH | | |
| (days) | 7.4 | 5.5 | 4.0 | 7.4 | 5.5 | 4.0 | 7.4 | 5.5 | 4.0 |

phosphate buffer:

| | | | | | | | | | |
|---|---|---|---|---|---|---|---|---|---|
| 1 | 269 ± 57 | 249 ± 61 | 236 ± 34 | 236 ± 37 | 261 ± 31 | 245 ± 37 | 213 ± 37 | 211 ± 41 | 234 ± 38 |
| 2 | 235 ± 45 | 215 ± 36 | 246 ± 48 | 246 ± 29 | 255 ± 36 | 268 ± 39 | 212 ± 33 | 216 ± 35 | 204 ± 27 |
| 3 | 242 ± 38 | 247 ± 31 | 259 ± 48 | 249 ± 30 | 229 ± 21 | 251 ± 35 | 240 ± 25 | 241 ± 28 | 234 ± 23 |

10% serum:

| | | | | | | | | | |
|---|---|---|---|---|---|---|---|---|---|
| 1 | 209 ± 41 | 200 ± 73 | 369 ± 82 | 136 ± 20 | 158 ± 29 | 275 ± 38 | 148 ± 21 | 159 ± 27 | 252 ± 36 |
| 2 | 273 ± 60 | 245 ± 50 | 466 ± 59 | 141 ± 23 | 184 ± 28 | 383 ± 20 | 152 ± 16 | 180 ± 24 | 354 ± 35 |
| 3 | 272 ± 52 | 311 ± 67 | 453 ± 91 | 154 ± 25 | 221 ± 32 | 428 ± 31 | 148 ± 29 | 207 ± 29 | 354 ± 27 |

### Example 8

Interliposome fluorescence resonance energy transfer (FRET) (ex: 496 nm, em: 590 nm) was used to evaluate possible fusion among liposomes at decreasing pH values. Two populations of liposomes were prepared. One population contained NBD-lipids (energy donor) and rhodamine-lipids (energy acceptor) each at 0.5 mol % fraction. The other liposome population did not contain fluorophores. Liposome fractions from each population were mixed at equal volumes at various pH values and their fluorescence intensity was compared to samples containing only liposomes from the fluorescent liposome population. Liposome fusion increases the effective distances between fluorophores, resulting in lower emission intensities of the energy acceptor. The fluorescent intensities of samples were monitored over time and the ratios of the intensities of samples containing both liposome populations were normalized by the intensities of samples containing only the fluorescent liposomes to account for quenching or photo-bleaching effects unrelated to fusion.

For the equimolar DPPC- and DSPA-containing liposomes, no change in the FRET intensities was observed over time (four days), both in phosphate buffer and in 10% serum supplemented media for all pH values studied (7.4, 5.5, 4.0) suggesting that no fusion occurs even after liposome aggregation in media.

### Example 9

Zeta potential measurements verified decrease of the surface negative charge of liposome membranes with decreasing pH. To better characterize the discontinuities along the domain/non-domain interface, the pH-dependent release of fluorescent dextrans of larger sizes was also studied. The zeta potential of dextrans was measured at the pH values studied and was found to range between −14.1 and −6.5 mV, and −3.6 and −4.1 mV for 3 KDa and 10 kDa dextrans, respectively, suggesting that a release mechanism other than direct diffusion across the membrane transient discontinuities would be highly unlikely.

The Zeta potential of liposomes containing different fractions of the titratable domain-forming lipid DSPA was measured in phosphate buffer at different pH values (7.4, 5.5, and 4.0) using a Zetasizer ZS90 (Malvern instruments, Worcestershire, UK). Measurements were performed in 10 mM PBS with no salt. For these measurements, liposomes (1 mM total lipid) were prepared without grafted PEG-chains on their surface to allow for closer approach of the plane of shear to the physical surface of lipid membranes. Exclusion of PEGylated phosphatidyl ethanolamine (pKa=1.7) at 2% mole fraction is not expected to significantly influence the values of zeta potential (except, probably, for the lower concentration (10% mole) of DSPA).

Table 5 shows the measured zeta potential values of liposomes containing different fractions of the titratable domain-forming lipid DSPA at pH 7.4, 5.5 and 4.0. Liposomes exhibited decreasing values of negative zeta potential with decreasing pH and with decreasing DSPA content.

TABLE 5

| | 50% mole DSPA | 25% mole DSPA | 10% mole DSPA |
|---|---|---|---|
| pH | Zeta potential (mV) | Zeta potential (mV) | Zeta potential (mV) |
| 7.4 | −68.5 ± 0.5 | −50.4 ± 0.6 | −41.5 ± 0.4 |
| 5.5 | −65.2 ± 0.7 | −41.7 ± 0.5 | −37.1 ± 0.5 |
| 4.0 | −55.3 ± 0.5 | −38.6 ± 0.7 | −32.1 ± 0.2 |

### Example 10

Dynamic light scattering (DLS) of liposome suspensions was studied with an N4 plus autocorrelator (Beckman-Coulter, Fullerton, Calif.), equipped with a 632.8 nm He—Ne laser light source. Scattering was detected at 23.0, 30.2, 62.6 and 90°. Particle size distributions at each angle were calculated from autocorrelation data analysis by CONTIN. The average liposome size was calculated to be the y-intercept at zero angle of the measured average particle size values versus sin²(θ). All buffer solutions used were filtered with 0.22 μm filters just before liposome preparation. The collection times for the autocorrelation data were 1 to 10 minutes. Liposomes were incubated in different solutions (phosphate buffer and serum supplemented media) at 37° C., and liposome fractions were removed from the parent liposome suspension over time, and were diluted in phosphate buffer (7.4) before measurement.

This example discloses liposome membranes composed of two types of lipids (DPPC and DSPA with 5% mole cholesterol) that exhibit lipid phase-separation as a response to decreasing pH values and resulting in content release. However, the presence of these relatively large discontinuities, these liposomes retain their structure in solution and do not collapse into larger aggregates as shown by DLS measurements. These studies qualitatively correlate the extent of non-ionized domain-forming DSPA lipids to the extent of lipid phase separation (measured by DSC). Increased incubation times resulted in higher calorimetric peaks (higher overall enthalpy changes) that potentially indicates increased number of lipids associated with the phase-separated domains or the formation of clustered phase-separated domains. Clustering of domains decreases the total domain/non-domain interface and should result in decrease of the number of thermal contributions of lower thermal transition temperatures that originate from lipids residing in the less ordered domain/non-domain interface.

### Example 11

Unilamellar PEGylated liposomes containing lipid pairs having dipalmitoyl (n=16) and distearoyl (n=18) chain lengths (both lipid T_{g}'s>37° C.) were made using the extrusion method and were verified by cryo-TEM. One lipid type was chosen to have the titratable moiety phosphatidic acid (PA) on its headgroup, and the other lipid type to have a phosphatidylcholine (PC) headgroup.

The effect of pH on the formation of lipid heterogeneous domains and membrane permeability was evaluated on vesicles composed of lipid pairs with matching and non-matching chain lengths.

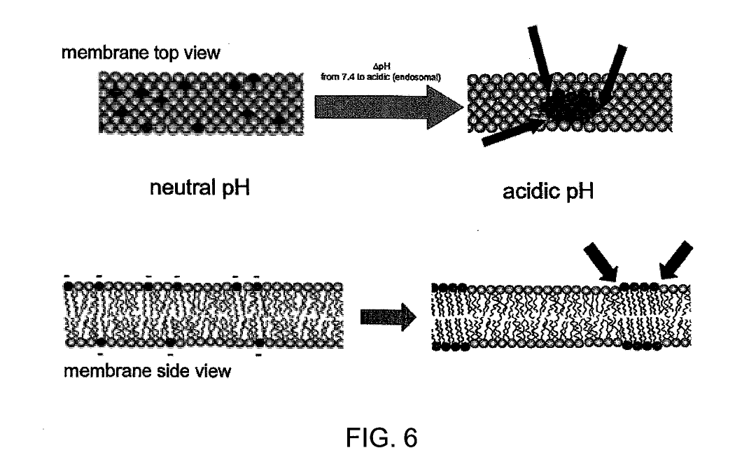

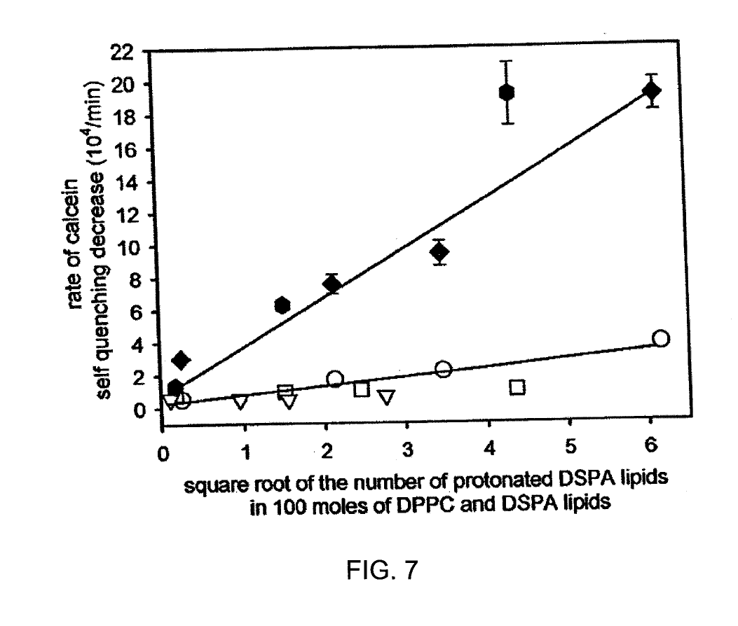

As provided herein, and as shown schematically in FIG. 6, formation of heterogeneous domains with decreasing pH from 7.4 to 4.0 does not significantly influence the permeability of membranes by itself in membranes comprising a rigid lipid component and a fluid lipid component with non-matching (different) chain lengths. Instead, membrane permeability was found to increase as the pH decreased by rigid liposomes comprising rigid lipid pairs with non-matching (different) chain lengths and in membranes comprising rigid lipid pairs with matching chain lengths, thereby suggesting defective packing among acyl chains of lipids at the domain interfaces among non-matching and matching chains. The membrane permeability rates were demonstrated to linearly depend on the total perimeter of the calculated interfacial boundaries of heterogeneous domains. As can be seen in FIG. 7, in the simplest approximation of small circular domains of similar size rich in protonated DSPA lipids, the total perimeter of interfacial boundaries should be proportional to the square root of the number of protonated DSPA lipids. The pKa value of 4.5 was used for phosphatidic acid to calculate the fraction of protonated DSPA lipids. For all lipid fractions, the rate of content release attributed to discontinuities at the interfacial boundaries is linearly proportional to the square root of protonated DSPA lipids and therefore proportional to the total perimeter of the discontinuities' interface (open symbols). For comparison, identical lipid membranes lacking cholesterol (closed symbols) exhibit similar dependence on the square root of protonated DSPA lipids but with faster release rates.

The pKa value of 4.5 is the lowest value above the reported value of 4.0 that results in arrangement of all data points in a straight line. This deviation of the pKa value can be attributed to several factors. First, the proposed model of small, circular domains is certainly an oversimplification. Second, because the apparent pKa of PA should depend on the surface charge density, the values of apparent pKa should be composition-dependent. In the presence of serum proteins, liposomes exhibit faster release rates with decreasing pH compared to the release rates in phosphate buffer. DSC studies suggest a second mechanism of lipid separation due to adsorption of charged proteins causing clustering of charged PA lipids via electrostatic attractions, which explains the faster release rates observed in the presence of serum proteins.

### Example 12

### Evaluation in More Realistic Conditions (Serum Supplemented Media)

At pH 7.4, any liposome composition containing DPPC lipids resulted in less than about 50% retention of contents after a 24 hour incubation in 60% serum supplemented media, which is the media solution used for cell culture containing serum proteins, at 37° C.

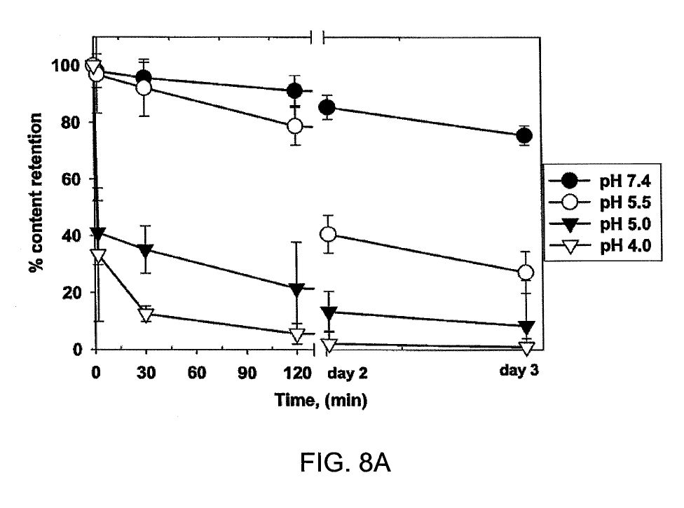

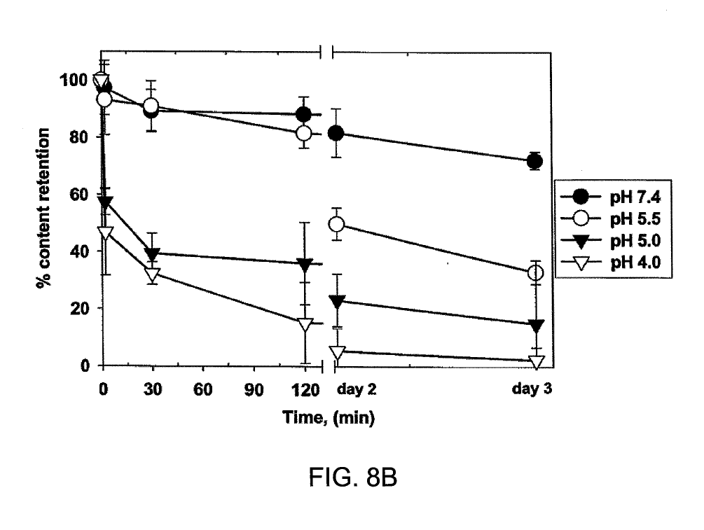

Therefore, liposomes were developed based on the same molecular principles of rigid lipids with PC and PA headgroups, and non-matching acy-tails, but with longer hydrocarbon chain lengths (n=18 and 21). The content release profiles of the liposome composition containing 21PC and DSPA at about a 75:25 mole ratio with about 10 mol % cholesterol, and about 15 mol % DSPE-PEG(2000) (21PC/DSPA-based liposome) in about 60% and about 10% serum supplemented media at 37° C. are shown on FIGS. 8A and 8B, respectively. As provided in FIGS. 8A and 8B, the 21PC/DSPA-based liposomes exhibit at a pH of about 7.4 stable retention of about 80% of contents for at least about three days, and at a pH of about 5.0 fast release of at least about 60% of their contents within about 30 minutes (pH 7.4 (); 5.5 (◯); 5.0 (▾); 4.0 (∇)).

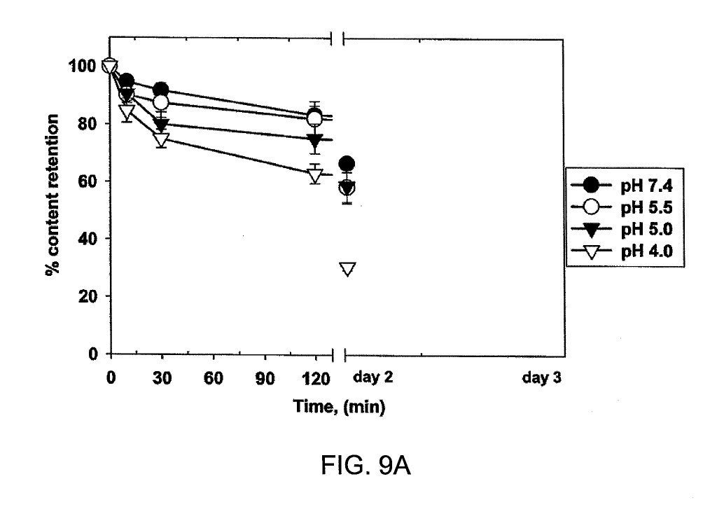

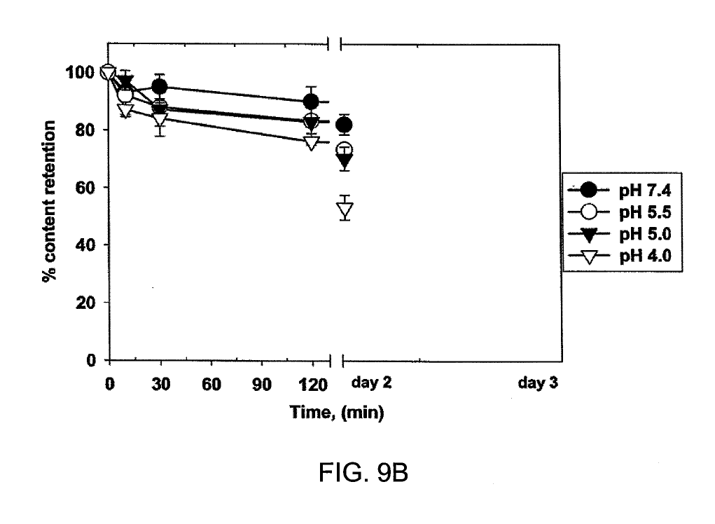

For comparison, FIG. 9A and FIG. 9B show that DSPC-based liposomes (90 mol % DSPC, 10 mol % cholesterol, and 5 mol % DSPE-PEG(2000)) that are similar to DOXIL®, (Ortho biotech, Bridgewater, N.J.) composition retain at a pH of about 7.4, 5.5 and 5.0 at least about 70% of contents for at least about 24 hours. At a pH of about 4.0, the DSPC-based liposomes release a maximum of about 40% of contents after about two hours.

To evaluate the release of calcein, vesicles containing self-quenching concentrations of calcein (55 mM) were incubated in phosphate buffer or serum supplemented media at different pH values at 37° C. over time. The concentration of lipids for incubation was 0.20 mM. Release of calcein from vesicles and its dilution in the surrounding solution results in increase in fluorescence due to relief of self-quenching (Allen, T. M.; et al., Serum induced leakage of liposome contents, Biochim Biophys Acta, 597, 418-426 (1980)). Calcein release was measured at different time points by adding fixed quantities of vesicle suspensions into cuvettes (with cross section 1 cm×1 cm) containing phosphate buffer (1 mM EDTA, pH 7.4). Calcein fluorescence (ex: 495 nm, em: 515 nm) before and after addition of Triton-X 100, was measured using a Fluoromax-2 spectrofluorometer (Horiba Jobin Yvon, NJ), and was used to calculate the quenching efficiency defined as the ratio of fluorescence intensities after and before addition of Triton-X 100. The percentage of retained contents with time was calculated as before:

$$\% calcein content retention = ( \frac{{Q}_{t} - {Q}_{min}}{{Q}_{max} - {Q}_{min}} ) \times 100$$

where, Qₜ is calcein quenching efficiency at the corresponding time point t, Qₘₐₓ is the maximum calcein quenching efficiency in phosphate buffer (at pH 7.4) at room temperature immediately after separation of vesicles by SEC, and Qₘᵢₙ is the minimum quenching efficiency equal to unity. This experimental approach may underestimate the actual retention of contents. Relief of calcein self-quenching occurs essentially at concentrations below 10 mM that is not significantly smaller than the initial concentration of the encapsulated fluorophore at 55 mM.

### Example 13

### Evaluation In Vitro

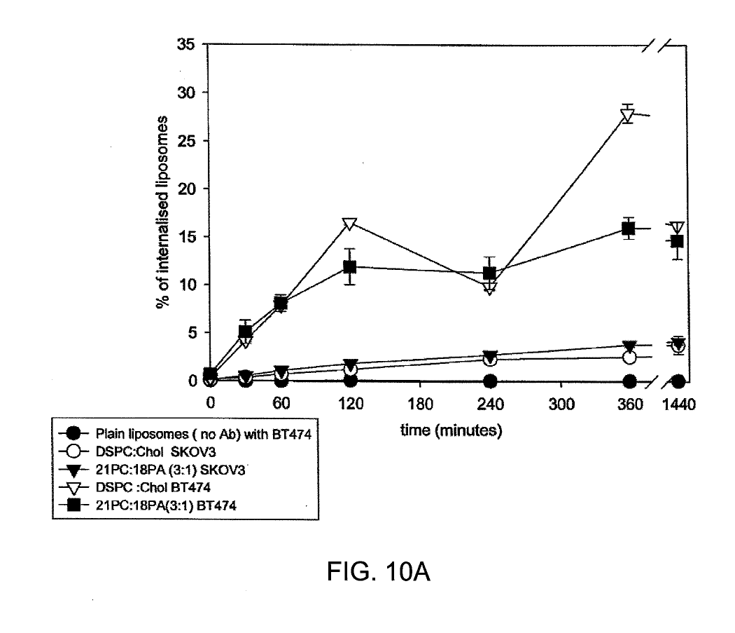

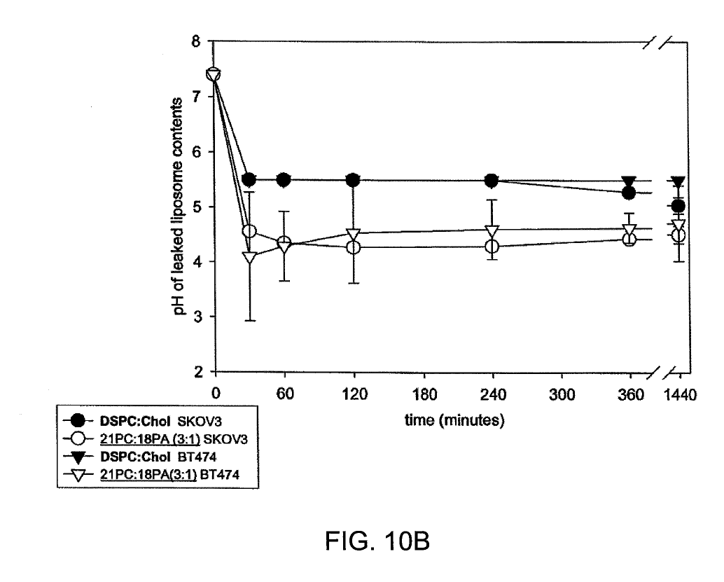

As provided herein, the effective pH-triggered content release at pH 5.0 is retained by the 21PC/DSPA-based liposomes in vitro. In particular, the extent of binding to SKOV3 and BT-474 cancer cells of Herceptin-labeled 21PC/DSPA-based liposomes was compared to Herceptin-labeled DSPC-based liposomes. The extent of immuno-liposomes bound per cell, and fraction of internalized immuno-liposomes was similar for both liposome compositions for the particular cell type, as determined by standard assay as shown in Sofou, S., et al., Enhanced Retention of the alpha-Particle-Emitting Daughters of Actinium-225 by Liposome Carriers, Bioconjugate Chem., 18, (6), 2061-2067 (2007). These results were consistent with both liposome formulations being of the same size and containing similar grafting densities of conjugated antibodies on their surface. See FIG. 10A.

However, a significant difference between the times required for the liposomes to release their contents into the endoplasm was observed. In these studies a membrane impermeant, fluorescent pH-indicator (calcein) was encapsulated into liposomes (at pH 7.4) that were used on the binding/internalization assay disclosed above. Upon immunoliposome internalization, and trypsinization of bound, non-internalized liposomes, the fluorescent excitation spectra of the contents of liposomes in cell suspensions were acquired, and the pH of the released contents was determined. Calcein exhibits pH-sensitive fluorescence emission profiles that can be used to identify the pH of the calcein's surrounding solution. As provided in FIG. 10B, the endocytosed 21PC/DSPA-based liposomes require less than about 30 minutes to release their contents at pH values of about 4.5, thereby suggesting that content release occurred during the acidification of the endosomal pathway in both cell lines (open symbols). The DSPC-based liposomes release their contents at a significantly lower extent, since the recorded collective pH of released and non-released contents did not decrease below the value of about pH 5.5 over the incubation of about 24 hours (closed symbols). In these studies, the fluorescent pH-indicator was encapsulated in the liposomes at pH=7.4.

These studies indicate superior kinetics of release of encapsulated contents in vitro of the 21PC/DSPA-based liposomes compared to DSPC-based liposomes. Therefore, the 21PC/DSPA-based liposomes can be employed for fast and efficient content release and increase the bioavailability of a therapeutic agent.

### Example 14

### Evaluation In Vivo

Biodistributions were evaluated in BALB/C female mice without tumor for 21PC/DSPA-based liposomes and for DSPC- and HSPC-based PEGylated liposomes. The HSPC-based PEGylated liposomes were chosen based on their similarity to FDA approved compositions. Liposomes encapsulating DTPA, a chelator to keep that radionuclides inside the liposomes, were loaded with ¹¹¹Indium using the ionophore A23187. For the protocol see Chang, M.-Y., et al., Enhanced Loading Efficiency and Retention of 225Ac in Rigid Liposomes for Potential Targeted Therapy of Micrometastases, Bioconjugate Chem., 19, (6), 1274-1282 (2008). The fraction of anionic lipid DSPA was altered, and the fraction of PEGylated lipids (DSPE-PEG) was adjusted in order to maximize the blood circulation times of the developed liposomes without compromising their pH-sensitivity. The AUC's of DSPC- and HSPC-based liposomes were also evaluated by dissecting animals at the indicated time points, and by removing blood, and all organs, weighting them, and then measuring their radioactivity following the gamma emissions of Indium-111, and were used for comparison.

Table 6 below shows the areas under the curve (AUC) for the blood clearance of all liposomes tested calculated from the % ID/gr plots. Animals were sacrificed at 1, 5, 24, and 72 hours post administration of liposomes via tail vein injection. Normal organ uptake was comparable for all compositions with liver and spleen being the major normal organs of accumulation. As provided in Table 6, two compositions which provide long circulation times and retain their pH-dependent content release are shown in bold font (numbered (3) and (4)). Their pH-dependence has been verified in vitro, as disclosed above.

Similar blood AUC suggests that the 21PC/DSPA-based liposomes have the same probability to extravasate from the blood into the tumor interstitium as the DSPC- and HSPC-based liposomes resulting in the same tumor absorbed doses. In addition to this, the 21PC/DSPA-based liposomes exhibit potentially greater ‘bioavailability’ in the sense that they will release the free drug intracellularly in a pH-dependent manner. Therefore, for the same therapeutic effect, lower administered doses will be required when 21PC/DSPA-based liposomes are used resulting in lower toxicities to normal organs.

TABLE 6

| | | Diameter, | Blood |
|---|---|---|---|
| Composition | mol ratio | nm | AUC |
| (1) DSPC:Cholesterol:DSPE-PEG | 70:30:5 | 143 ± 10 | 338 |
| (2) HSPC:Cholesterol:DSPE-PEG | 61:39:5 | 152 ± 13 | 242 |
| (3) 21PC:DSPA:DSPE-PEG | 90:10:5 | 137 ± 9 | 210 |
| 21PC:DSPA:Cholesterol:DSPE-PEG | 75:25:10:5 | 141 ± 12 | 129 |
| DSPC:Cholesterol:DSPE-PEG | 70:30:15 | 147 ± 12 | 183 |
| HSPC:Cholesterol:DSPE-PEG | 61:39:15 | 154 ± 9 | 194 |
| 21PC:DSPA:Cholesterol:DSPE-PEG | 83:17:10:15 | 149 ± 16 | 368 |
| (4) | 75:25:10:15 | 140 ± 6 | 212 |
| 21PC:DSPA:Cholesterol:DSPE-PE | | | |

### Example 15

Evaluation in more realistic conditions: incubation in 60% serum proteins and demonstration of pH-dependent release of encapsulated doxorubicin.

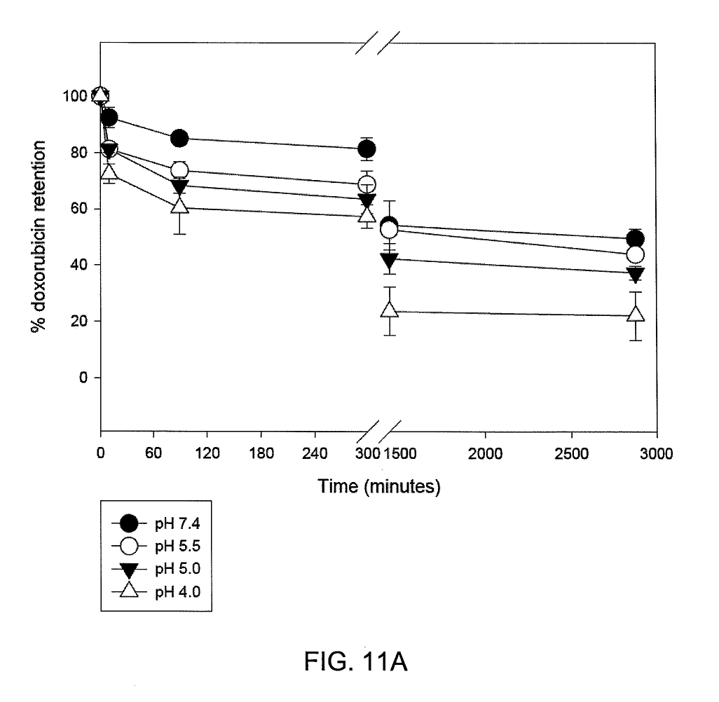

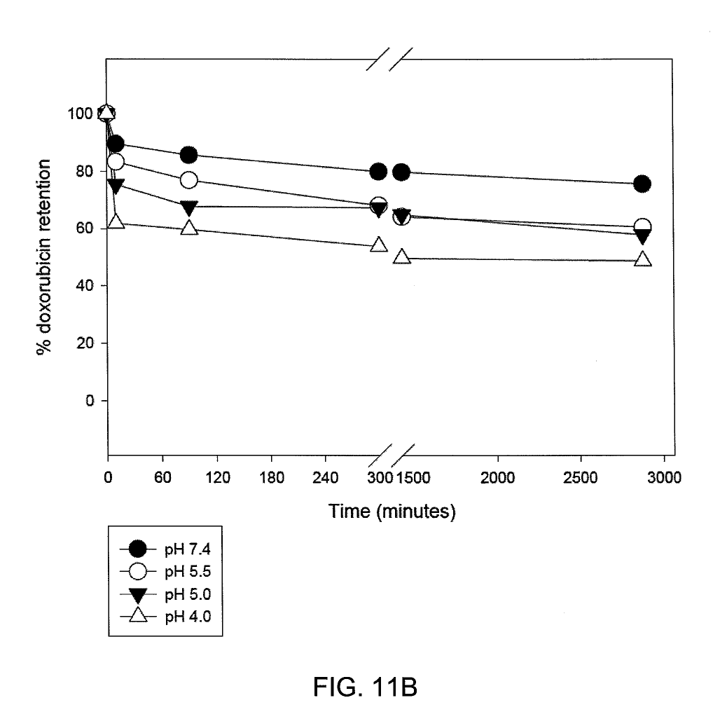

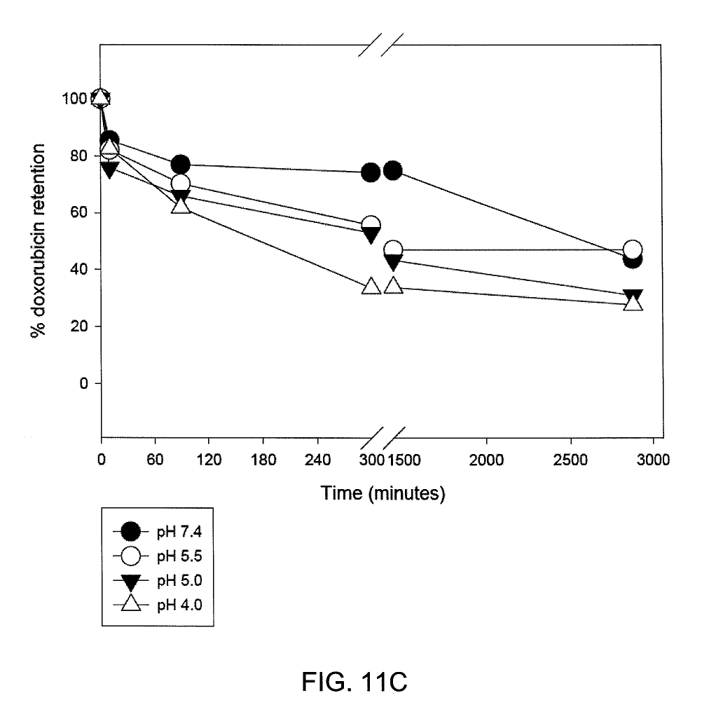

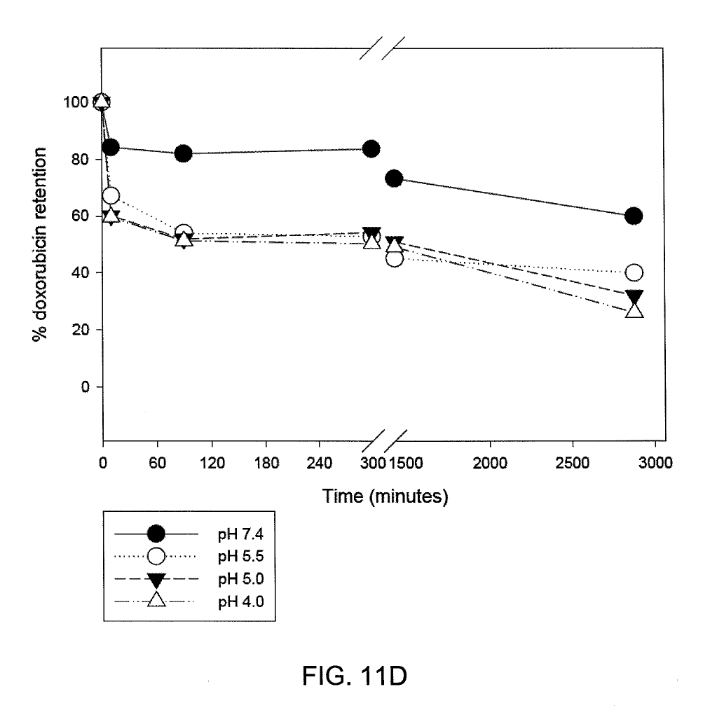

Currently, liposomes, and in particular liposomes with encapsulated doxorubicin, are approved for the therapy of cancer (DOXIL®/CAELYX®) mainly due to improvement of the pharmacokinetics (PK) of the delivered drug by reducing its acute and chronic cardiotoxicity. However, these liposomes are not designed to exhibit triggered release within the cancer cells. Therefore doxorubicin's bioavailability within the cancer cells that constitute the tumors depends on the natural enzymatic (and other) mechanisms for slow destabilization of the liposome membrane and slow content release. Luckily, doxorubicin escapes degradation by lysosomal enzymes. FIG. 11 illustrates a new generation of liposomes that are tuned to have release rates synchronized with the cancer cells' physiology, while also exhibiting the improved PK profiles for doxorubicin exhibited by USFDA (United States Food and Drug Administration) approved liposomes. These liposomes exhibit pH-dependent release of encapsulated doxorubicin [pH 7.4 (); 5.5 (◯); 5.0 (▾); 4.0 (∇)].

Some of the liposome compositions with long circulation times shown in bold in Table 6 and numbered (3) (FIG. 11C) and (4) (FIG. 11D) were loaded with doxorubicin using published loading protocols [see Haran, G., et al., “Transmembrane ammonium sulfate gradients in liposomes produce efficient and stable entrapment of amphipathic weak bases” Biochimica et Biophysica Acta, 1151 (1993) 201-215] and were compared to DSPC:Cholesterol:DSPE-PEG (FIG. 11A) and HSPC:Chol:DSPE-PEG (FIG. 11B) liposome formulations numbered in Table 6 as (1) and (2), respectively. The two latter compositions are similar to USFDA approved lipid compositions for the liposomal doxorubicin drug named DOXIL®/CAELYX®. All liposome compositions exhibit pH-dependent release of encapsulated doxorubicin. However, liposome formulations (3) (FIG. 11C) and (4) (FIG. 11D) exhibit not only the same stable retention of doxorubicin at the physiological pH 7.4 that corresponds to pH conditions during circulation in the blood, but they also release fast and more extensively the encapsulated doxorubicin compared to the USFDA approved compositions (1) (FIG. 11A) and (2) (FIG. 11B). In particular, after one hour of incubation at the early endosomal pH of 5.5 and 5.0, 50% of the initially encapsulated drug is released from the 21PC:DSPA:Cholesterol:DSPE-PEG (liposome numbered (4) in Table 6) as shown in FIG. 11D. DSPC:Cholesterol:DSPE-PEG and HSPC:Chol:DSPE-PEG liposome formulations numbered in Table 6 as (1) and (2) release less than 30% of the initial drug content after the same incubation time and identical pH conditions. Liposomes composed of 21PC:DSPA: DSPE-PEG numbered (3) in Table 6 and shown in FIG. 11C exhibit 70% drug release which is the highest observed after 5 hours of incubation at pH 4 at 37° C. in the presence of 60% serum proteins.

To evaluate the release of doxorubicin, vesicles containing doxorubicin were incubated in 60% serum supplemented media at different pH values at 37° C. over time. The concentration of lipids for incubation was 0.20 mM. Release of doxorubicin from vesicles was measured at different time points by separating from liposomes the leaked doxorubicin by size exclusion chromatography using a Sephadex G-50 column (of 11 cm length) eluted with phosphate buffer (1 mM EDTA, pH=7.4). Triton-X 100 was added to the collected eluent corresponding to liposomally encapsulated doxorubicin that was heated at 85° C. for 10 minutes to effectively release encapsulated doxorubicin. The sample was allowed to cool at room temperature before measuring the fluorescent intensity that is proportional to doxorubicin using a Fluoromax-2.

The foregoing detailed description of the embodiments and the appended figures have been presented only for illustrative and descriptive purposes. They are not intended to be exhaustive and are not intended to limit the scope and spirit of the invention. The embodiments were selected and described to best explain the principles of the invention and its practical applications. One skilled in the art will recognize that many variations can be made to the invention disclosed in this specification without departing from the scope and spirit of the invention.

What is claimed is:

1. A liposome composition containing a biologically active agent, comprising at least two types of lipids that phase separate in at least two domains formed by:
i) a zwitterionic first lipid having a hydrophobic tail and a head group that when zwitterionic is substantially miscible;
ii) a second lipid having a hydrophobic tail and a titratable charged head group, that, when protonated, is substantially immiscible with the first lipid;
iii) a polyethyleneglycol-linked lipid having a side chain matching at least a portion of the first or the second lipid;
iv) cholesterol; and
v) an entrapped biologically active agent;
wherein the composition is adapted to release the entrapped at a certain pH.

2. The liposome composition of claim 1, wherein said liposomes further include a stabilizing component.

3. The liposome composition of claim 1, wherein the first lipid is a phosphatidylcholine lipid.

4. The liposome composition of claim 1, wherein the first and second lipids are present in equal proportions.

5. The liposome composition of claim 1, wherein the first and second lipids are present in unequal proportions.

6. The liposome composition of claim 1, wherein the first and second lipids each have Tg>37° C.

7. The liposome composition of claim 1, wherein the certain pH is lower that about 7.

8. The liposome composition of claim 1, further comprising a coating on the membrane surfaces of the liposomes that preferentially associate with a specific target cell.

9. The liposome composition of claim 1, wherein the biologically active agent is toxic to cancer cells.

10. The liposome composition of claim 1, wherein the head group of one of the lipids is negative at a neutral pH.

11. The liposome composition of claim 1, wherein the first lipid is DSPC, DPPC, or 21PC and the second lipid is DSPA.

12. The liposome composition of claim 1, wherein the mole ratio of the first lipid to the second lipid ranges from about 75-90:10-25.

13. The liposome composition of claim 1, wherein the mole % of the third lipid is about 10-20.

14. The liposome composition of claim 1, further comprising about 5-15 mole % of cholesterol.

15. The liposome composition of claim 1, comprising about 83 mole % of a phosphatidylcholine lipid having a 21 hydrocarbon tail, about 17 mole % DSPA, about 10 mole % cholesterol, and about 15 mole % DSPE-PEG.

16. The liposome composition of claim 1, comprising about 75 mole % of a phosphatidylcholine lipid having a 21 hydrocarbon tail, about 25 mole % DSPA, about 10 mole % cholesterol, and about 15 mole % DSPE-PEG.

17. A method for treating a malignant tumor in a mammal comprising administering to the mammal a pharmaceutically effective amount of the liposome composition of claim 1.

18. The method as claimed in claim 17, wherein the head group on the second lipid is selected so that the liposome composition will release the biologically active agent at a desired pH.

19. A method for increasing accumulation of a biologically active agent proximal to a cell having a acidic environment, comprising:
a) administering liposomes comprising at a least first lipid and a second lipid each having a head group and hydrophobic tail, a polyethyleneglycol-linked lipid having a tail matching at least a portion of the first or the second lipid, and an entrapped biologically active agent, wherein the first lipid is a zwitterionic lipid and the second lipid has a titratable head group, and the liposome are adapted to release the entrapped at a certain pH; and
b) allowing the liposomes to release the biologically active agent in the acidic environment,
whereby the release of the biologically active agent is effective to achieve at accumulation of the biologically active agent in the acidic environment.

20. The method as claimed in claim 19, wherein the liposomes are capable of releasing the biological agent at metastatic tumors with developed vasculature.

21. A method for administering a biologically active agent comprising:
a) selecting a liposome comprising at a least first rigid lipid and a second rigid lipid each having a head group and a hydrophobic tail and a polyethyleneglycol-linked lipid having a side chain matching at least a portion of the first or the second lipid, wherein the first lipid is a zwitterionic lipid and the second lipid has a titratable head group;
b) preparing a liposome composition with the at least the first rigid lipid and the second rigid lipid and the polyethyleneglycol-linked lipid;
c) preparing a therapeutic liposome by combining the composition with a biologically active agent so that the biologically active agent is within the liposome composition whereby the therapeutic liposome is adapted to release the entrapped at a certain pH; and
d) administering the therapeutic liposome to a subject.

22. The method as claimed in claim 21, wherein the liposome composition is prepared to release the biologically active agent in an environment with a pH of 5.5.

23. The method as claimed in claim 21, wherein the first and the second lipid are selected so that the liposome composition will release the biologically active agent at a desired pH.

24. The method as claimed in claim 21, wherein the head group on the first lipid is selected so that the liposome composition will release the biologically active agent at a desired pH.
